(19) United States
(12) Patent Application Publication
(10) Pub. No.: US 2026/0245656 A1
(43) Pub. Date: Aug. 20, 2026

(54) METHOD TO PREDICT IMMUNOGENIC DOUBLE-STRANDED RNA BURDEN

(71) Applicant: The Board of Trustees of the Leland Stanford Junior University, Stanford, CA (US)

(72) Inventors: Jin Billy Li, Stanford, CA (US); Qin Li, Redwood City, CA (US)

(21) Appl. No.: 18/869,767

(22) PCT Filed: Jun. 7, 2023
(86) PCT No.: PCT/US2023/024697

§ 371 Date: Nov. 26, 2024

Related U.S. Application Data
(60) Provisional application No. 63/473,678, filed on Jun. 7, 2022.

(51) Int. Cl. G16B 20/20; G16B 25/00

(52) U.S. Cl. CPC G16B 20/20; G16B 25/00

(57) ABSTRACT

Compositions and methods are provided for determining immunogenic dsRNA level in an individual, based on the individual's genotype. This information is useful for disease stratification, and for selecting appropriate therapeutic intervention, including, for example inhibition of MDA5, inhibition of type 1 interferon signaling, and the like. In some embodiments a tissue specific subset of immunogenic dsRNAs are analyzed, where the tissue is associated with an immune-related disease of interest.

### BACKGROUND

Genome-wide association studies (GWAS) have led to the discovery of hundreds of thousands of risk variants involved in trait and disease etiology, but understanding their molecular function remains an ongoing challenge. Quantitative trait loci (QTL) studies, best exemplified by gene expression QTLs (eQTLs), have been successful in bridging GWAS variants to their molecular mechanisms. Alternative splicing QTLS (sQTLs) have further expanded discovery of these mechanisms. However, other post-transcriptional processes, such as RNA editing, remain largely unexplored, despite the increasing appreciation of their important functions in health and disease.

One of the most abundant RNA modifications is adenosine-to-inosine (A-to-I) RNA editing catalyzed by adenosine deaminases acting on RNA (ADARs) that bind double-stranded RNA (dsRNA) substrates and convert adenosines to inosines. Since inosine is recognized as guanosine, RNA editing events can be accurately identified and quantified by standard RNA sequencing, unlike most other RNA modifications. Previous studies have identified millions of RNA editing sites in humans, >99% of which are located in inverted repeat Alus (IRAlus) that form dsRNA substrates.

Key to editing in mammals are two enzymatically active ADAR proteins, ADAR1 and ADAR2, which have distinct physiological functions in vivo. ADAR1, ubiquitously expressed across human tissues, plays a critical role to suppress dsRNA sensing that is mediated by MDA5, a cytosolic sensor of “non-self” dsRNA (FIG. 1a). Mice deficient with Adar1 editing are embryonic lethal due to elevated innate immune responses indicated by the induction of interferon-stimulated genes (ISGs), but can be rescued to full life span when MDA5 is knocked out.

Immunogenic double-stranded RNA (dsRNA) structures are endogenous dsRNA structures that resemble viral RNA and may trigger false activation of the innate immune response, leading to severe damage to the host cell. Adenosine to inosine (A-to-I) RNA editing is a common post-transcriptional modification, abundant within repetitive elements of all metazoans. A key function of A-to-I RNA editing by ADAR1 is to suppress the immunogenic response by endogenous dsRNAs.

In humans, ADAR1 loss-of-function and MDA5 gain-of-function mutations have been identified in rare autoimmune diseases such as Aicardi-Goutieres Syndrome (AGS), further establishing the ADAR1-dsRNA-MDA5 axis as an underlying mechanism in immune disease (FIG. 1a). Protective, loss-of-function, alleles in MDA5 have also been found in GWAS of common inflammatory diseases such as type-1 diabetes (T1D), psoriasis, inflammatory bowel disease, vitiligo, vitamin B12 deficiency anaemia, hypothyroidism and coronary artery disease. Further, aberrant editing has been reported in several common autoimmune diseases including psoriasis, rheumatoid arthritis (RA), systemic lupus erythematosus (SLE) and multiple sclerosis (MS). However, the extent to which common genetic differences in RNA editing contribute to immune and inflammatory diseases remains to be identified.

### SUMMARY

Compositions and methods are provided for determining immunogenic dsRNA level in an individual, based on the individual's genotype. This information is useful for disease stratification, and for selecting appropriate therapeutic intervention, including, for example inhibition of MDA5, inhibition of type I interferon signaling, and the like. In some embodiments a tissue specific subset of immunogenic dsRNAs are analyzed, where the tissue is associated with an immune-related disease of interest.

Increased immunogenic dsRNA level is strongly associated with a higher risk for autoimmune and inflammatory diseases. It is shown herein that ADAR-mediated adenosine-to-inosine (A-to-I) RNA editing is a primary mechanism underlying the association of certain genetic variants with common autoimmune and immune-related diseases. Cis RNA editing QTLs, which are referred to herein as “edQTLs”, are identified herein across multiple human tissues. An exemplary listing of such edQTLs is provided in Supplementary Table 3 of Li et al. (2022) Nature 608:569-577. These edQTLs are significantly enriched in GWAS signals for autoimmune and immune-mediated diseases.

Also identified are dsRNA loci of disease relevance, which may be defined as dsRNAs whose sufficient editing is important to suppress autoimmunity. Determining signal colocalization between edQTLs found in 49 human tissues, and reported genetic variants obtained from GWAS studies, identified colocalization events linking immune-related diseases to genes expressing putative immunogenic dsRNAs. The majority of these immunogenic dsRNAs are located in exons, specifically in UTRs where long dsRNA structures are often formed.

Immunogenic dsRNAs act in aggregate to elicit cellular immunogenicity. Risk variants of these immune-related diseases show directional effects, where across multiple autoimmune and immune-related diseases, there is an overwhelmingly negative direction of effects, i.e., risk GWAS variants lead to less dsRNA editing in general. The directional effects of RNA editing are even more significant when tested in tissue types of disease relevance. Associations are shown for autoimmune diseases including, for example, autoimmune thyroid disease (ATD), celiac disease, inflammatory bowel disease (IBD), primary biliary cirrhosis (PBC), systemic lupus erythematosus (SLE), multiple sclerosis, vitiligo, psoriasis, type 1 diabetes (T1D), rheumatoid arthritis; for atopic conditions such as asthma and atopic dermatitis; and for other conditions with a significant inflammatory component, e.g. amyotrophic lateral sclerosis (ALS), coronary artery disease, Parkinson's disease, systemic sclerosis, schizophrenia, Alzheimer's disease, and levels of high and low density lipoproteins and triglycerides.

In some embodiments, methods are provided for determining an individual's immunogenic dsRNA score (IDS), which score is correlated with immune-related disease, and which can be predictive of response to therapy, e.g. therapy directed at reducing the clinical sequelae of undesirable activity in dsRNA sensing pathways. The IDS can also be used in the stratification of individuals for clinical trials, and to identify individuals responsive to therapy that reduces the clinical sequelae of undesirable activity in dsRNA sensing pathways. Such undesirable activity may include, without limitation, decreased ADAR-mediated RNA editing systemically or in targeted tissues; increased MDA5 activity systemically or in targeted tissues; and increased type I interferon expression systemically or in targeted tissues. These activities provide points of therapeutic intervention for individuals determined to be at risk. In some embodiments the individual has been previously disgnosed with an immune-related disease of interest.

In some embodiments a method of determining an individual's immunogenic dsRNA score (IDS) comprises genotyping said subject at a plurality of risk alleles associated with undesirable activity in dsRNA sensing pathways, and generating a double-stranded RNA (dsRNA) burden score by weighting the number of risk alleles that decrease RNA editing levels by (a) the effect size of association with the disease, and (b) the effect size of their association with RNA editing level. The risk alleles may be selected from SNPs listed in Supplementary Table 3 of Li et al. (2022) Nature 608:569-577; which information is also provided in Table 2 of the priority provisional application U.S. 63/473,678, herein specifically incorporated by reference. A subset of the SNP list is provided in Supplementary Table 3 of Li et al. (2022) Nature 608:569-577; which information is also provided in Table 3 of the priority provisional application U.S. 63/473,678, herein specifically incorporated by reference.

In some embodiments the immunogenic dsRNA score determination further comprises genotyping the individual at one or more alleles associated with MDA5 or ADAR1. In some embodiments the immunogenic dsRNA score further comprises determining the expression level of dsRNAs in disease related tissues or cells derived therefrom . . .

In some embodiments the immunogenic dsRNA score further comprises inputting additional clinical indicia, including without limitation one or more of: age, gender, ethnic group, genetic background, family history, age of onset of said disease, duration of said disease, and the like.

Genotyping can be performed on a DNA sample from the individual for single-nucleotide polymorphisms, e.g. by sequencing, hybridization, etc. as known in the art. The methods disclosed herein identify a subset of SNP loci, for example as set forth in Supplementary Table 3 of Li et al. (2022) Nature 608:569-577, herein specifically incorporated by reference, that predict immunogenic dsRNA levels by quantitative trait loci (QTL) mapping in a plurality of individuals. DNA sequencing is used to classify the SNP loci that have a p-value score below a pre-set threshold, with a directional effect that increases immunogenic dsRNA level. Training on genome-wide association study (GWAS) data is used to assess distribution of IDS between cases and controls for a given autoimmune/inflammatory disease, and determine the IDS thresholds to generate predictions for high risk for the disease. The methods may include genotyping of at least about 100, at least 200, at least about 500, at least about 1000, at least about 5000, at least about 10,000, at least about 15,000, at least about 20,000, at least about 25,000, at least about 30,000 SNPs, and may genotype substantially all of the edQT SNPs listed in Supplementary Table 3 of Li et al. (2022) Nature 608:569-577.

The immunogenic dsRNA score (IDS) can be calculated based on factors as set forth above. For a given individual, the predicted IDS of the disease relevant tissue/cell-type higher than the IDS thresholds for a given disease is considered as indicative of high risk. In some embodiments the IDS (W) is determined by the equation:

$${Ψ}^{k} = {\sum }_{i = 1 , j = 1}^{n} {β}_{j} \times {ϕ}_{ij}^{k} \times {ε}_{i}^{k}$$

where

$${β}_{j}^{k}$$

is the effect size of j^{th} SNP for the disease,

$${ϕ}_{ij}^{k}$$

is the effect size of i^{th} dsRNA with j^{th} SNP for edQTL in k^{th} tissue/cell type and

$${ε}_{ij}^{k}$$

is the expression level of i^{th} dsRNA in k^{th} tissue/cell type, which may be measured, for example by can be measured by qPCR, RNA-seq, scRNA-seq, etc.; and optionally further comprises clinical indicia as described above.

Many diseases have an underlying inflammatory component that contributes to disease initiation and/or progression. In some embodiments, the methods comprise treating an individual in accordance with the IDS determination. Diseases of interest include, for example, autoimmune diseases such rheumatoid arthritis (RA), T1D, systemic lupus erythematosus (SLE), multiple sclerosis (MS), autoimmune hepatitis; degenerative diseases such as osteoarthritis (OA), Alzheimer's disease (AD), and macular degeneration; metabolic diseases including type II diabetes, metabolic syndrome, non-alcoholic steatohepatitis (NASH), and alcoholic steatohepatitis; cardiovascular diseases such as atherosclerosis; cancers which can arise from and induce inflammation; as well as other diseases with an inflammatory component such as Parkinson's disease, Alzheimer's disease and the like.

The methods disclosed herein include steps of data analysis, which may be provided as a program of instructions executable by computer and performed by means of software components loaded into the computer. Such methods include one or more of inputting genotyping data, e.g. sequence information for SNP loci of interest; inputting effect size of an SNP for disease association, inputting effect size of an SNP for RNA editing level association, inputting expression levels of dsRNA in tissues of interest. The methods can also include determination of threshold levels for a disease by training on GWAS data is used to assess distribution of IDS between cases and controls for a given disease, and determining the IDS thresholds to generate predictions for high risk for the disease. The methods can include solving the equation

$${Ψ}^{k} = {\sum }_{i = 1 , j = 1}^{n} {β}_{j} \times {ϕ}_{ij}^{k} \times {ε}_{i}^{k}$$

for these inputs. Other bioinformatics methods are provided for determining and quantitating when the IDS is physiologically relevant. The method may further comprise providing a computer-generated report comprising the analysis and determination of an IDS.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1: RNA editing QTL map in human tissues. a, Schematic graph of the ADAR1-dsRNA-MDA5 axis in innate immunity. b, Illustration of cis-edQTL concept. RNA editing levels were quantified from bulk RNA-seq of tissue samples by counting the fraction of edited reads over edited and unedited reads for each editing site, and local genetic effects (edQTLs) were quantified across individuals for each tissue. c, The number of edSites mapped in GTEx tissues, compared to the number mapped in LCLs from a previous study. Each tissue is color-coded according to its overall editing level. Tissues of notably high or low overall editing level are labeled. Dashed line represents linear regression between the number of edSites and sample size. d, Pairwise sharing of edQTLs across tissues. Colors indicate Pearson's correlation coefficient by the magnitude of edQTL effect size. Tissues with notably distinctive edQTL profiles are labeled. e, Comparison of the distribution of SNPs mapped as eQTLs, sQTLs or edQTLs around gene bodies (top panel), splice junctions (middle panel) and RNA editing sites (bottom panel). The density of SNPs is normalized to [0,1] within each panel. Standard deviation of SNP density is shown as a shaded area. f, Alignment of ADAR1 binding profile with edQTL SNPs in the Alu consensus sequence. ADAR1 binding profile was estimated using ADAR1 CLIP-seq data (Methods). SNPs are colored by local density (window size=10 nt). f′, Correlation between the binding preference of ADAR1 and the absolute effect sizes of edQTLs. We divided the 300 nt Alu consensus sequence into 60 equal bins and within each bin, we tested the correlation between the average absolute effect sizes of SNPs and the average ADAR1 binding preference using Spearman's method. Error bars represent standard deviation.

FIG. 2: Contribution of edQTLs to complex diseases and traits. a, Quantile-quantile (Q-Q) plots of p-values for GWAS SNPs annotated as edQTLs, sQTLs, and eQTLs. Random SNPs matching the edQTLs are used as negative controls (Methods). b, Enrichment of heritability for 24 human traits and diseases mediated by edQTLs, sQTLs and eQTLs. Autoimmune diseases are shown in bold. Enrichment is measured as the regression coefficient of MESC (Methods). Error bars represent standard deviation. c, Estimated percentage of heritability mediated by edQTLscross GTEx tissues for 24 human traits and diseases. Traits and diseases are shown in the same order as in b. Tissues with common biological origin are put into tissue groups (top row, see Methods).

FIG. 3: Identification, characterization and validation of disease-relevant, putatively immunogenic dsRNAs. a, Fractions of GWAS significant loci explained by edQTLs. 17 autoimmune and immune-related diseases were ranked by the fraction of GWAS hits colocalized with edQTLs. The total number of colocalized loci is shown for each disease. Distribution of genomic location is shown for putatively immunogenic dsRNAs (b) and all dsRNAs with edSites (b′). c, Putatively immunogenic dsRNA loci colocalized with multiple autoimmune or immune-related diseases. Gene names of putatively immunogenic dsRNA loci are shown for each group. d, LocusZoom plots of the top edQTL locus colocalized with IBD GWAS. Signals for IBD GWAS, edQTLs, eQTLs and sQTLs are shown from top to bottom, respectively. The extent of LD with the lead SNP in GWAS (rs1886731, purple rhombus) is shown by a gradient of color. d′, Evidence supporting the dsRNA formation. Zoom-in plot shows the dsRNA associated with the lead causal SNP. Opposite transcriptional directions of TNFRSF14 and TNFRSR14-AS1 are supported by gene annotation (top row) and by stranded RNA-seq (middle row) with plus- and minus-strand reads plotted separately. Formation of dsRNA in the overlapped region between two genes is supported by hyper-editing (bottom row, each red bar representing an editing site). e, Validation of dsRNA formation in GTEx tissues. Expression levels of TNFRSF14 and TNFRSR14-AS1 are shown for GTEx tissues in which at least one of the pair is expressed. The formation of dsRNA in each tissue is verified by RNA editing signal (Methods). H4PP scores are shown for validation of putative causal effects of the cis-NATs in IBD GWAS. f, Fraction of putatively immunogenic dsRNAs formed by IRAlus or non-repetitive (cis-NATs) in immune-related diseases. Diseases with at least 6 colocalized dsRNAs loci are shown for comparison. Data for body mass index (bottom) is shown as a control. g, Comparative analysis of dsRNAs formed by IRAlus and cis-NATs. We tested three dsRNA features key to MDA5 sensing: the length of dsRNAs measured by the base-pairing stem length (left), overall double-strandedness measured by the sequence identity of stem region (middle), and the fraction of hyper-edited adenosines in the dsRNAs (right). P-values were calculated using Mann-Whitney U test. h, Negative stain electron microscopy images of the MDA5 filaments along the unedited (left) and edited (right) dsRNAs formed by TNFRSF14 and TNFRSR14-AS1. h′, Filament length comparison of unedited and edited dsRNAs using two-sided unpaired Student's t-test. i, Real-time PCR measurements of three representative ISGs (CXCL10, OAS2 and ISG15) in ADAR15912A cells with Dox-inducible MDA5, transfected with plasmids expressing CSTA:PLTP dsRNA (Unedited), CSTA:PLTP dsRNA with scrambled sequences (Scrambled), or single-stranded RNA (ssRNA control). Plasmids expressing CSTA:PLTP dsRNA were also transfected into ADAR1WT cells with Dox-inducible MDA5 (Edited). Expression fold-changes of three representative ISGs were calculated by the ratio between cells treated with and without Dox that induces MDA5. n=3, *****, p<0.00001; n.s. p>0.5 (Wilcoxon signed-rank test). Error bars represent standard error.

FIG. 4: Risk variants of inflammatory diseases collectively reduce nearby dsRNA editing levels. a, Schematic diagram showing that risk variants, by reducing editing levels of immunogenic dsRNAs, lead to MDA5-mediated innate immunity. Genome-wide directional effects of risk variants on (b) editing levels and (c) expression levels observed in IBD. Effect sizes estimated through SLDP regression are shown on x-axis to denote the overall direction of effects of risk variants on editing level, and are plotted against genome-wide significance (y-axis) for each GTEx tissue. Tissues showing significant directional effects are colored in red (Bonferroni corrected p-value <1e-3). d, Risk variants reduce RNA editing levels and induce ISG expression in rheumatoid arthritis. RNA-seq data from 152 patient-derived samples (synovial tissues) were used to calculate the overall editing levels of dsRNAs associated with risk vs. protective alleles defined by GWAS (see Methods). Each sample is colored by IFN scores calculated using expression levels of signature ISGs from the same RNA-seq samples (see Methods). e, Immune signature scores of the rheumatoid arthritis samples grouped by the extent of reduced editing levels (protective-risk alleles) in quantiles, from low to high. Expression of predefined signature genes for IFN, MHC-I, TGFb and PD1/PDL1 immune pathways were used to calculate the scores for each sample. Statistical tests were performed using one-way ANOVA for the association between each immune signature score and editing level differences, respectively. ****, P<0.0001; n.s., P >0.05. f, Schematic diagram illustrating RNA editing of immunogenic dsRNAs bridges the gap between risk variants and susceptibility of autoimmune and immune-related diseases.

FIG. 5. Single-cell gene expression analysis of disease samples identifies the cell types of high IFN response, potentially as a result of dsRNA-mediated MDA5 sensing. a, UMAP plots showing IFN scores of pancreatic islet cells in non-diabetic controls (left) and T1D donors (right). The single-cell data was downloaded from the Human Pancreas Analysis Program (HPAP). b, UMAP plots showing IFN scores of coIon epithelial cells in non-IBD controls (left) and IBD donors (right). The single-cell data was from Kong et al. (2023) Immunity 56 (2): 444-458. c, UMAP plots showing IFN scores of carotid artery cells from patient-matched proximal adjacent portions of carotid artery tissue (left) and calcified atherosclerotic core plaques (right). The single-cell data was from Alsaigh et al. (2022) Communications Biology 5 (1): 1084.

FIG. 6. Single-cell gene expression analysis of disease samples identifies the cell types expressing high levels of immunogenic dsRNAs. a, UMAP plots showing the aggregated expression of non-immunogenic dsRNAs (left) and immunogenic dsRNAs (right) in pancreatic islet cells. The single-cell data was downloaded from the Human Pancreas Analysis Program (HPAP). b, UMAP plots showing the aggregated expression of non-immunogenic dsRNAs (left) and immunogenic dsRNAs (right) in coIon epithelial cells. The single-cell data was from Kong et al. (2023) Immunity 56 (2): 444-458. c, UMAP plots showing the aggregated expression of non-immunogenic dsRNAs (left) and immunogenic dsRNAs (right) in carotid artery cells. The single-cell data was from Alsaigh et al. (2022) Communications Biology 5 (1): 1084.

FIG. 7. Prediction of IFN score by dsRNA burden score. a, Predicted dsRNA burden scores correlate with IFN scores measured directly in IBD RNA-seq samples. The IBD samples were from Haberman et a. (2019) Nature Communication 10 (1): 38. b, Predicted dsRNA burden scores correlate with IFN scores measured directly in CAD RNA-seq samples. The prediction of dsRNA burden score was performed on 213 individuals of the GTEx consortium. The plot shows positive correlation between the predicted dsRNA burden scores with IFN scores (R²=0. 62, p-value=3e-4) at the individual level. For both panels a and b, the dsRNA burden score was based on individual genetic information, combining the known genetic effects on dsRNA editing level (edQTLs) in disease-relevant tissues and the known average expression level of immunogenic dsRNA genes, to make the prediction. We also controlled for population structure, age, and sex in the final scores. The IFN scores were quantified using the expression level of interferon-stimulated genes (ISGs) measured in the RNA-seq.

FIG. 8. Quantification of RNA editing levels in GTEx data. a, Number of editing sites used for cis-edQTL mapping in each tissue. Editing sites mapped uniquely by >20 reads in ≥60 samples of each tissue type were considered. Both GTEx V6p and V8 results are plotted here for comparison. b, Comparison of RNA editing levels quantified using exact read count versus estimated read count for cis-NAT editing sites (Methods). c, Percentage of editing variance across individuals explained by top 10 principal components (PCs) of editing level. d, Association between ADAR1 expression level with PC 1 of editing level. e, Density plot of editing level measurements and editing level variance between individuals (n=175; normalized standard deviation) of 123,707 sites in Brain-cerebellar hemisphere tissue. Editing sites detected in ≥60 individuals were used for this analysis.

FIG. 9. Identification of RNA editing QTLs across GTEx tissues. a, Fraction of editing sites (left) and edited genes (right) found with edQTLs in each tissue. The LCLs data was obtained from previous edQTL study. b, Fractions of edQTLs shared between multiple editing sites. c, Fractions of edGenes having multiple independent edQTLs. Each of the 49 tissues was tested and plotted individually in b and c. d, Exemplary locus of TRMT9B showing two independent edQTLs each regulating multiple editing sites. The two edQTLs are represented by their lead SNPs rs13268982 and rs34995506, respectively. Genome browser view of the TRMT9B 3′ UTR that consists of four IRAlus (shown in the 1st track from top), with editing sites (2nd track from top), SNP location (black bars) and variant-editing association effect sizes for each editing site (3rd and 4th tracks) shown from top to bottom. Effect sizes are colored by direction of effects: red for positive effect and blue for negative effect. e, Comparison of the number of edSites identified in this study and in Park et al., 2021. For each tissue type, edSites are compared between two studies by evaluating (1) whether they are tested for genetic association, and (2) whether the associations are significant under different p-value cut-offs (p<1e-3 on the left, p<1e-5 on the right). f, Sharing of edQTLs across tissues by sign (same direction of effects). Euclidean distance matrix across tissues was calculated for hierarchical clustering using Ward's method. g, Fraction of edQTLs shared by the number of tissues according to sign. g′, Fraction of edQTLs shared by the number of tissues according to magnitude. Effects with more than 2-fold change of sizes from one another are considered as of different magnitudes. h, Number of tissue-specific edQTLs effects found in 20 representative tissues. Representative tissues were picked by two criteria: 1) large sample size (≥150); 2) least number of shared editing sites with other tissue. For example, a high number of editing sites are shared between three arteries tissues so we choose Artery-Coronary tissue as the representative one since it has the largest sample size. i, Sharing of edQTLs with eQTLs and sQTLs. We estimated the fraction of shared QTLs according to Storey's TT1 (Methods). SNPs with matching numbers and allele frequencies with the edQTLs were randomly sampled genome-wide in each tissue to be used as the control set. j, Distance between the best edQTL and best eQTL for genes with both types of QTL, using 1,478 genes in whole blood tissue as an example. k, Enrichment of functional elements underlying edQTLs, eQTLs and sQTLs (left to right). We used a Bayesian hierarchical model to identify putatively causal variants driving a QTL from a set of variants associated with the locus, and quantified the enrichment of strongly associated variants in functional elements. For edQTLs, we additionally identified the functional elements underlying edQTL SNPs that are >800 bp away from the edSites (grey dots). We used chromHMM annotations and gene-level annotations (e.g. intronic variant, splice region variants, UTR variants, etc.) from snpEff. Splice region variants include variants located within the region of splice site (1-3 bases of the exon side or 3-8 bases of the intron side) and branch point. Structured RNA are defined as regions with icSHAPE score >0.7 in RNA structure mapping data in vivo from multiple cell lines. ADAR1 and other RBP binding sites are defined using CLIP peaks.

FIG. 10. Characterization of edQTLs' effects on RNA sequences and secondary structures. a, Q-Q plot of edQTLs annotated with distance from the associated editing sites. b, Summarized edQTL effect sizes of alternative alleles by 4 types of RNA ribonucleotides at each position from −50 to +50 nt relative to the editing site. Stronger effects were observed for ±2 and ±1 positions and illustrated to show the “AUAGG” motif centered at the edited “A”. c, Further breakdown of the effects of 12 different nucleotide changes (reference allele->alternative allele) caused by SNPs located at ±2 and ±1 positions. Reference alleles (y-axis) and alternative alleles (x-axis) were accounted for by their transcribed ribonucleotides on the RNA. d, An exemplary edSite association showing negative effects of C-to-U change at ±2 position of editing site. Editing site at chr1: 184761188 and SNP at chr1: 184761186 are presented here (hg38 coordinates). Predicted RNA secondary structures for reference allele (G on DNA and C on RNA) and alternative allele (A on DNA and U on RNA) are shown on the left, with editing level measurements of different alternative allele dosages on the right. e, Schematic diagram showing RNA secondary structural features defined by bpRNA. f, Frequency of edQTL SNPs found in different structural features compared to non-QTL SNPs in the same predicted structure. Statistical significance was calculated using the Mann-Whitney U test.

FIG. 11. RNA editing QTLs are enriched in immune-related diseases and immune traits. a, QQ-plots of IBD, MS, RA and CAD GWAS with QTL annotations. The expression levels of eGenes and sGenes were matched to the levels of edGenes within ±15% of deviation (see Methods). b, Enrichment of heritability for 42 human traits and diseases mediated by edQTLs, sQTLs and eQTLs. Autoimmune diseases are shown in bold. Enrichment is measured as the regression coefficient of MESC (Methods). Error bars represent standard deviation. Meta-analysis of RNA editing QTL mediated heritability is shown in c and heritability enrichment in c′, for 4 exemplary autoimmune and immune-related diseases. Tissue groups are defined the same way as in FIG. 2c. Tissues and tissues groups shown in c′ are the same as in c. Error bars represent standard deviation.

FIG. 12. Fraction of SNP heritability (h²g) mediated by edQTL in immune-related trait GWAS. We performed MESC analysis on 33 GWAS data obtained from Sayaman et al. in the same way as in FIG. 2C. We filtered out 11 immune traits with low total SNP heritability (h²g <0.01) from the final result. The remaining 22 traits are categorized and colored by immune function annotations according to Sayaman et al. Dashed line indicates 0.1 of mediated SNP heritability (h²g).

FIG. 13. Directional effects of risk variants associated with complex traits and diseases on RNA editing levels. a, Traits and diseases with overall negative effects on RNA editing (increased risk associated with reduced editing level). b, Traits and diseases with overall positive or unclear direction of effects on RNA editing. For each disease or complex trait, we plotted the genome-wide-log 10 (P) against estimated effect size for each GTEx tissue using SLDP regression. Significant tissues are colored in red (Bonferroni corrected p-value <1×10⁻³). Larger circles denote lower p-values. c, Directional effect analysis of four exemplary diseases for edQTLs (top row), eQTLs (middle row) and control edQTLs with randomly flipped signs (bottom row).

FIG. 14. Risk genetic variants are associated with reduced RNA editing levels and induced IFN score in inflammatory diseases. RNA-seq data from patient-derived samples of three diseases (multiple sclerosis, lupus and CAD) were used to calculate the overall editing levels of dsRNAs associated with risk vs protective alleles and the IFN scores. We used allele-specific editing analysis to determine editing levels associated with risk vs protective alleles (a, b and c). The IFN scores were calculated by summing up the expression of representative ISGs for each disease (Methods), and grouped in four different bins. Samples are then grouped by their editing level differences (protective alleles-risk alleles, divided by quartiles) to show IFN scores plotted for each group and compared between groups of the same disease type (a′, b′ and c′). ***, P<0.001; **, P<0.01; one-way ANOVA test.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

An SNP (single-nucleotide polymorphism) is a germline substitution of a single nucleotide at a specific position in the genome. Within a population, SNPs can be assigned a minor allele frequency—the lowest allele frequency at a locus that is observed in a particular population. Association studies can be used to determine whether a genetic variant is associated with a disease or trait. One of main contributions of SNPs in clinical research is genome-wide association study (GWAS). Genome-wide genetic data can be generated by multiple technologies, including SNP array and whole genome sequencing. GWAS has been commonly used in identifying SNPs associated with diseases or clinical phenotypes or traits. Since GWAS is a genome-wide assessment, a large sample site is required to obtain sufficient statistical power to detect all possible associations. Some SNPs have relatively small effect on diseases or clinical phenotypes or traits. To estimate study power, the genetic model for disease needs to be considered, such as dominant, recessive, or additive effects. Bioinformatics databases for SNPs include, for example, dbSNP from the National Center for Biotechnology Information (NCBI); the Human Gene Mutation Database; the International HapMap Project, GWAS Central, etc.

GWAS. Genome-wide association studies (GWAS) broadly test for differences in the allele frequency of genetic variants between individuals who are ancestrally similar but differ phenotypically. GWAS can consider copy-number variants or sequence variations in the human genome, although the most commonly studied genetic variants in GWAS are single-nucleotide polymorphisms (SNPs). GWAS typically report blocks of correlated SNPs that all show a statistically significant association with the trait of interest, known as genomic risk loci. GWAS have shown that most traits are influenced by thousands of causal variants that individually confer very little risk, are often associated with many other traits and are correlated with causal and non-causal variants that are physically close as a result of linkage disequilibrium.

The workflow of a GWAS involves the collection of DNA and phenotypic information from a group of individuals; genotyping of each individual using available GWAS arrays or sequencing strategies; quality control; imputation of untyped variants using haplotype phasing and reference populations; conducting the statistical test for association; and include interpreting the results by conducting multiple post-GWAS analyses.

QLT. Quantitative trait locus analysis is a statistical method to link phenotypic and genotypic data. Several types of markers can be used, including single nucleotide polymorphisms (SNPs), simple sequence repeats (SSRs, or microsatellites), restriction fragment length polymorphisms (RFLPs), and transposable element positions. Markers that are genetically linked to a QTL influencing the trait of interest will segregate more frequently with trait values, whereas unlinked markers will not show significant association with phenotype.

Public databases can be used in QTL analysis, for example the Genotype-Tissue Expression (GTEx) project includes data from samples collected from 54 non-diseased tissue sites across nearly 1000 individuals, primarily for molecular assays including WGS, WES, and RNA-Seq. Remaining samples are available from the GTEx Biobank. The GTEx Portal provides open access to data including gene expression, QTLs, and histology images. GTEx data can be mapped against genotype data, e.g. SNP data.

eQTL are specific regions of the genome that are associated with the variation in gene expression levels. eQTL analysis aims to identify and map the genetic loci that influence the expression levels of genes. These loci can be genetic variants, such as single nucleotide polymorphisms (SNPs), that are found within or near genes and affect the regulation or activity of the genes. By studying eQTL, researchers can gain insights into the genetic factors that contribute to differences in gene expression between individuals and understand how these variations may impact complex traits and diseases.

In the present disclosure, QLT analysis is used to account for RNA editing levels, defined as the fraction of edited (‘G’) transcripts over total (‘A’ and ‘G’) transcripts at single nucleotides across a catalogue of sites. To identify and control for potential confounding factors of editing level measurement, principal component analysis (PCA) may be performed.

Identification of edQTLs in a tissue type may utilize the QTL mapping pipeline adopted by the GTEx consortium as known in the art, for example see Liang et al. (2021) Nature Communications volume 12, article 1424; Wang et al. (2021) BMC Bioinformatics. 22 (Suppl 9): 403; THE GTEX CONSORTIUM (2020) Science 369 (6509): 1318-1330; each herein specifically incorporated by reference.

Genotyping. A number of methods are used for determining the presence of a variant in an individual. Genomic DNA is isolated from the individual or individuals that are to be tested. DNA can be isolated from any nucleated cellular source such as blood, hair shafts, saliva, mucous, biopsy, feces, etc. Methods using PCR amplification can be performed on the DNA from a single cell, although it is convenient to use at least about 10⁵ cells. Where large amounts of DNA are available, the genomic DNA can be used directly.

The methods of the present disclosure can involve sequencing target loci, as well as analyzing sequence data. Various methods and protocols for DNA sequencing and analysis are well-known in the art and are described herein. Sequencing can be accomplished using high-throughput systems. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read. Sequencing can be performed using nucleic acids described herein such as genomic DNA, cDNA derived from RNA transcripts or RNA as a template. Sequencing may comprise massively parallel sequencing.

For example, DNA sequencing may be accomplished using high-throughput DNA sequencing techniques. Examples of next generation and high-throughput sequencing include, for example, massively parallel signature sequencing, polony sequencing, 454 pyrosequencing, Illumina (Solexa) sequencing with HiSeq, MiSeq, and other platforms, SOLID sequencing, ion semiconductor sequencing (Ion Torrent), DNA nanoball sequencing, heliscope single molecule sequencing, single molecule real time (SMRT) sequencing, MassARRAY®, and Digital Analysis of Selected Regions (DANSR™). See, e.g., Stein RA (1 Sep. 2008). “Next-Generation Sequencing Update”. Genetic Engineering & Biotechnology News 28 (15); Quail, Michael; Smith, Miriam E; Coupland, Paul; Otto, Thomas D; Harris, Simon R; Connor, Thomas R; Bertoni, Anna; Swerdlow, Harold P; Gu, Yong (1 Jan. 2012). “A tale of three next generation sequencing platforms: comparison of Ion torrent, pacific biosciences and illumina MiSeq sequencers”. BMC Genomics 13 (1): 341; Liu, Lin; Li, Yinhu; Li, Siliang; Hu, Ni; He, Yimin; Pong, Ray; Lin, Danni; Lu, Lihua; Law, Maggie (1 Jan. 2012). “Comparison of Next-Generation Sequencing Systems”. Journal of Biomedicine and Biotechnology 2012:1-11; Qualitative and quantitative genotyping using single base primer extension coupled with matrix-assisted laser desorption/ionization time-of-flight mass spectrometry (MassARRAY®). Methods Mol Biol. 2009; 578:307-43; Chu T, Bunce K, Hogge WA, Peters DG. A novel approach toward the challenge of accurately quantifying fetal DNA in maternal plasma. Prenat Diagn 2010; 30:1226-9; and Suzuki N, Kamataki A, Yamaki J, Homma Y. Characterization of circulating DNA in healthy human plasma. Clinica chimica acta; international journal of clinical chemistry 2008; 387:55-8). Similarly, software programs for primary and secondary analysis of sequence data are well-known in the art.

In some embodiments, high-throughput sequencing involves the use of technology available by Helicos BioSciences Corporation (Cambridge, Massachusetts) such as the Single Molecule Sequencing by Synthesis (SMSS) method. In some embodiments, high-throughput sequencing involves the use of technology available by 454 Lifesciences, Inc. (Branford, Connecticut) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

In some embodiments, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication application Nos. 200401061 30; 20030064398; 20030022207; and Constans, A, The Scientist 2003, 17 (13): 36.

In some embodiments, high-throughput sequencing of RNA or DNA can take place using AnyDot.chips (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication application No. 20030044781 and 2006/0078937. The growing of the nucleic acid strand and identifying the added nucleotide analog may be repeated so that the nucleic acid strand is further extended and the sequence of the target nucleic acid is determined.

The methods disclosed herein may include amplification of DNA. Amplification may comprise PCR-based amplification. Alternatively, amplification may comprise nonPCR-based amplification. Amplification of DNA may comprise using bead amplification followed by fiber optics detection as described in Marguiles et al. “Genome sequencing in microfabricated high-density pricolitre reactors”, Nature, doi: 10.1038/nature03959; and well as in US Publication Application Nos. 200200 12930; 20030058629; 20030 1001 02; 20030 148344; 20040248 161; 200500795 10,20050 124022; and 20060078909.

Amplification of the nucleic acid may comprise use of one or more polymerases. The polymerase may be a DNA polymerase. The polymerase may be a RNA polymerase. The polymerase may be a high fidelity polymerase. The polymerase may be KAPA HiFi DNA polymerase. The polymerase may be Phusion DNA polymerase.

Of interest is the use of the polymerase chain reaction (PCR) to amplify the DNA that lies between two specific primers. The use of the polymerase chain reaction is described in Saiki et al. (1985) Science 239:487, and a review of current techniques may be found in McPherson et al. (2000) PCR (Basics: From Background to Bench) Springer Verlag; ISBN: 0387916008. A detectable label may be included in the amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′, 7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. 32P, 35S, 3H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Primer pairs are selected from the genomic sequence using conventional criteria for selection. The primers in a pair will hybridize to opposite strands, and will collectively flank the region of interest. The primers will hybridize to the complementary sequence under stringent conditions, and will generally be at least about 16 nt in length, and may be 20, 25 or 30 nucleotides in length. The primers will be selected to amplify the specific region suspected of containing the predisposing mutation. Typically the length of the amplified fragment will be selected so as to allow discrimination between repeats of 3 to 7 units. Multiplex amplification may be performed in which several sets of primers are combined in the same reaction tube, in order to analyze multiple exons simultaneously. Each primer may be conjugated to a different label.

The exact composition of the primer sequences are not critical to the invention, but they must hybridize to the flanking sequences under stringent conditions. Criteria for selection of amplification primers are as previously discussed. To maximize the resolution of size differences at the locus, it is preferable to choose a primer sequence that is close to the SNP sequence, such that the total amplification product is at least about 30, more usually at least about 50, preferably at least about 100 or 200 nucleotides in length, which will vary with the number of repeats that are present, to not more than about 500 nucleotides in length. The number of repeats has been found to be polymorphic, as previously described, thereby generating individual differences in the length of DNA that lies between the amplification primers. Conveniently, a detectable label is included in the amplification reaction. Multiplex amplification may be performed in which several sets of primers are combined in the same reaction tube. This is particularly advantageous when limited amounts of sample DNA are available for analysis. Conveniently, each of the sets of primers is labeled with a different fluorochrome.

Effect size determination. For GWAS data, the effect sizes are usually reported in the summary statistics and expressed as either an odds ratio or a percentage of the phenotypic variance attributable to the locus (for quantitative traits such as weight and height). The effect size for an edQTL of RNA editing is defined as the slope of the linear regression between genotype dosages and RNA editing levels and is computed as the effect of the alternative allele relative to the reference allele (allele reported in the human genome reference sequence). The determination of effect size for an edQTL can be performed with statistical packages of QTL mapping, e.g. using FastQTL to perform a nominal pass followed by a permutation pass to determine all the associations between all genetic variants within a certain cis window (e.g. 100 kb) with the editing level of RNA editing sites at the center of the cis window.

Expression level analysis. The methods of the disclosure can include analysis of expression levels of transcripts of interest, e.g. expression of immunogenic dsRNAs. Any convenient method can be used for determining the level of a specific RNA in a sample. Methods of interest include, without limitation, microarray analysis, RNAseq, qPCR, and the like.

Microarrays hybridize an RNA population of interest or cDNA derived therefrom to a set of probes known as probes to determine the relative abundance of the mRNA in the target.

RNA-Seq, also known as whole transcriptome shotgun sequencing, attempts to perform the same function that DNA microarrays have been used to perform in the past, but with greater resolution. In particular, DNA microarrays utilize specific probes, and creation of these probes necessarily depends on prior knowledge of the genome and the size of the array being produced. RNA-seq removes these limitations by simply sequencing all of the cDNA produced in microarray experiments. This is made possible by next-generation sequencing technology. The technique has been rapidly adopted in studies of diseases like cancer [4]. The data from RNA-seq is then analyzed by clustering in the same manner as data from microarrays would normally be analyzed.

RNAseq is a sequencing technique that uses next-generation sequencing (NGS) to reveal the presence and quantity of RNA in a biological sample. In a typical workflow RNA are isolated from multiple samples, converted to cDNA libraries, sequenced into a computer-readable format, aligned to a reference, and quantified for downstream analyses such as differential expression. Single-cell RNA sequencing (scRNA-Seq) provides the expression profiles of individual cells. Patterns of gene expression can be identified through gene clustering analyses.

In real time PCR (qPCR), amplification of sequences is performed in the presence of a fluorescent due, fluorescence is measured after each cycle and the intensity of the fluorescent signal reflects the momentary amount of DNA amplicons in the sample at that specific time. In initial cycles the fluorescence is too low to be distinguishable from the background. However, the point at which the fluorescence intensity increases above the detectable level corresponds proportionally to the initial number of template DNA molecules in the sample. This point is called the quantification cycle and allows determination of the absolute quantity of target DNA in the sample according to a calibration curve constructed of serially diluted standard samples (usually decimal dilutions) with known concentrations or copy numbers. Strategies for the real time visualization of amplified DNA fragments include non-specific fluorescent DNA dyes and fluorescently labeled oligonucleotide probes.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

The term “sequence identity,” as used herein in reference to DNA sequences, refers to the sequence identity between two molecules. When a position in both of the molecules is occupied by the same monomeric nucleotide, then the molecules are identical at that position. The similarity between two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

Conditions that can be associated with IDS for prognosis, stratification and treatment treatment include a number of inflammatory conditions. Many diseases have an underlying inflammatory component that contributes to disease initiation and/or progression. Thus, the spectrum of inflammatory diseases and diseases associated with inflammation is broad and includes autoimmune diseases such rheumatoid arthritis (RA), systemic lupus erythematosus (SLE), multiple sclerosis (MS), and autoimmune hepatitis; degenerative diseases such as osteoarthritis (OA), Alzheimer's disease (AD), and macular degeneration; metabolic diseases including type II diabetes, metabolic syndrome, non-alcoholic steatohepatitis (NASH), and alcoholic steatohepatitis; cardiovascular diseases such as atherosclerosis; neurologic conditions such as amyotrophic lateral sclerosis (ALS), Parkinson's disease, Alzheimers disease, etc; as well as other diseases with an inflammatory component.

Rheumatoid Arthritis (RA) is a chronic syndrome characterized usually by symmetric inflammation of the peripheral joints, potentially resulting in progressive destruction of articular and periarticular structures, with or without generalized manifestations (Firestein (2003) Nature 423 (6937): 356-61; McInnes and Schett. (2011) N Engl J Med. 365 (23): 2205-19). The cause is unknown. A genetic predisposition has been identified, and, in some populations, localized to a pentapeptide in the HLA-DR beta1 locus of class II histocompatibility genes. Environmental factors may also play a role. For example, cigarette smoking places individuals possessing HLA-DR4 containing the “shared epitope” polymorphism at approximately 10⁻²⁰ fold increased risk of developing RA. Cigarette smoking is thought to induce anti-citrullinated protein antibody (ACPA) responses, which are measured using the commercial cyclic-citrullinated peptide (CCP) assay (Klareskog et al. (2006) Arthritis Rheum. 54 (1): 38-46). In addition, periodontitis and infection with P. gingivalis might also play a role in the initiation of autoimmune responses that result in development of RA (Rutger and Persson. 2012, J Oral Microbiol. 4). Immunologic changes may be initiated by multiple factors. About 0.6% of all populations are affected, women two to three times more often than men. Onset may be at any age, most often between 25 and 50 yr.

Systemic lupus erythematosus (SLE) is a systemic autoimmune disease characterized by malar rashes, oral ulcers, photosensitivity, serositis, seizures, low white blood cell counts, low platelet counts, seizures, a positive anti-nuclear antibody (ANA) test, and other positive autoantibodies. SLE is an autoimmune disease characterized by polyclonal B cell activation, which results in a variety of anti-protein and non-protein autoantibodies that result in immune complexes and inflammation which contributes to tissue damage (see, e.g., Kotzin et al., 1996, Cell 85:303-06 for a review of the disease). SLE has a variable course characterized by exacerbations and remissions and is difficult to study. For example, some patients may demonstrate predominantly skin rash and joint pain, show spontaneous remissions, and require little medication. The other end of the spectrum includes patients who demonstrate severe and progressive kidney involvement (glomerulonephritis and cerebritis) that requires therapy with high doses of steroids and cytotoxic drugs such as cyclophosphamide. Hydroxychloroquine slows SLE progression, and is a mainstay therapeutic for the management of SLE.

Multiple sclerosis (MS) is a debilitating, inflammatory, neurological illness characterized by demyelination of the central nervous system. The disease primarily affects young adults with a higher incidence in females. Symptoms of the disease include fatigue, numbness, tremor, tingling, dysesthesias, visual disturbances, dizziness, cognitive impairment, urological dysfunction, decreased mobility, and depression. Four types classify the clinical patterns of the disease: relapsing-remitting, secondary progressive, primary-progressive and progressive-relapsing (S. L. Hauser and D. E. Goodkin, Multiple Sclerosis and Other Demyelinating Diseases in Harrison's Principles of Internal Medicine 14th Edition, vol. 2, Mc Graw-Hill, 1998, pp. 2409-19).

Inflammatory bowel diseases, include Crohn's disease and ulcerative colitis, involve autoimmune attack of the bowel. These diseases cause chronic diarrhea, frequently bloody, as well as symptoms of colonic dysfunction.

Systemic sclerosis (SSc, or scleroderma) is an autoimmune disease characterized by fibrosis of the skin and internal organs and widespread vasculopathy. Patients with SSc are classified according to the extent of cutaneous sclerosis: patients with limited SSc have skin thickening of the face, neck, and distal extremities, while those with diffuse SSc have involvement of the trunk, abdomen, and proximal extremities as well. Internal organ involvement tends to occur earlier in the course of disease in patients with diffuse compared with limited disease (Laing et al. (1997) Arthritis. Rheum. 40:734-42). The majority of patients with diffuse SSc who develop severe internal organ involvement will do so within the first three years after diagnosis at the same time the skin becomes progressively fibrotic (Steen and Medsger (2000) Arthritis Rheum. 43:2437-44.). Common manifestations of diffuse SSc that are responsible for substantial morbidity and mortality include interstitial lung disease (ILD), Raynaud's phenomenon and digital ulcerations, pulmonary arterial hypertension (PAH) (Trad et al. (2006) Arthritis. Rheum. 54:184-91.), musculoskeletal symptoms, and heart and kidney involvement (Ostojic and Damjanov (2006) Clin. Rheumatol. 25:453-7). Current therapies focus on treating specific symptoms, but disease-modifying agents targeting the underlying pathogenesis are lacking.

Autoimmune hepatitis is a disease in which the body's immune system attacks liver cells. This immune response causes inflammation of the liver, also called hepatitis. Researchers think a genetic factor may make some people more susceptible to autoimmune diseases. About 70 percent of those with autoimmune hepatitis are female. The disease is usually quite serious and, if not treated, gets worse over time. Autoimmune hepatitis is typically chronic, meaning it can last for years, and can lead to cirrhosis—scarring and hardening-—of the liver. Eventually, liver failure can result.

Coronary artery disease (CAD): is a narrowing or blockage of the arteries and vessels that provide oxygen and nutrients to the heart. It is caused by atherosclerosis, an accumulation of fatty materials on the inner linings of arteries. The resulting blockage restricts blood flow to the heart. When the blood flow is completely cut off, the result is a heart attack. CAD is the leading cause of death for both men and women in the United States. Atherosclerosis (also referred to as arteriosclerosis, atheromatous vascular disease, arterial occlusive disease) as used herein, refers to a cardiovascular disease characterized by plaque accumulation on vessel walls and vascular inflammation. The plaque consists of accumulated intracellular and extracellular lipids, smooth muscle cells, connective tissue, inflammatory cells, and glycosaminoglycans. Inflammation occurs in combination with lipid accumulation in the vessel wall, and vascular inflammation is with the hallmark of atherosclerosis disease process.

Myocardial infarction is an ischemic myocardial necrosis usually resulting from abrupt reduction in coronary blood flow to a segment of myocardium. In the great majority of patients with acute MI, an acute thrombus, often associated with plaque rupture, occludes the artery that supplies the damaged area. Plaque rupture occurs generally in vessels previously partially obstructed by an atherosclerotic plaque enriched in inflammatory cells. Altered platelet function induced by endothelial dysfunction and vascular inflammation in the atherosclerotic plaque presumably contributes to thrombogenesis. Myocardial infarction can be classified into ST-elevation and non-ST elevation MI (also referred to as unstable angina). In both forms of myocardial infarction, there is myocardial necrosis. In ST-elevation myocardial infraction there is transmural myocardial injury which leads to ST-elevations on electrocardiogram. In non-ST elevation myocardial infarction, the injury is sub-endocardial and is not associated with ST segment elevation on electrocardiogram. Myocardial infarction (both ST and non-ST elevation) represents an unstable form of atherosclerotic cardiovascular disease. Acute coronary syndrome encompasses all forms of unstable coronary artery disease. Heart failure can occur as a result of myocardial dysfunction caused by myocardial infraction.

The presence of inflammation in a condition can be detected by a variety of approaches, including clinical history, physical examination, laboratory testing, histologic analysis of tissue, analysis of biomarkers, and imaging. Clinical features and physical exam markers of inflammation include swelling, effusions, edema, redness, warmth, pain, or associated pathologically with the influx of inflammatory cells or production of inflammatory mediators. Laboratory testing and/or histologic markers are abnormal when increased numbers of inflammatory cells are demonstrated. Markers of inflammation can include a molecular marker(s), and examples of a molecular marker(s) include C-reactive protein, a cytokine, an antibody, a DNA sequence, an RNA sequence, a cartilage marker, a metabolic marker, a bone marker, or combinations thereof. Imaging can reveal findings including enhancement of tissues, edema and swelling of tissues, and other findings indicative of inflammation. Examples of imaging markers of inflammation can include imaging markers measured using magnetic resonance imaging, ultrasound, computed tomography, angiography, and combinations thereof.

In some embodiments the methods of the invention comprise the step of identifying individuals “at-risk” for development of, or in the “early-stages” of, an inflammatory disease. “At risk” for development of an inflammatory disease includes: (1) individuals whom are at increased risk for development of an inflammatory disease, and (2) individuals exhibiting a “pre-clinical” disease state, but do not meet the diagnostic criteria for the inflammatory disease (and thus are not formally considered to have the inflammatory disease).

Individuals “at increased risk” for development (also termed “at-risk” for development) of an inflammatory disease are individuals with a higher likelihood of developing an inflammatory disease or disease associated with inflammation compared to the general population. Such individuals can be identified based on their IDS as defined herein, and can also include indicia including, without limitation: a family history of inflammatory disease; the presence of certain genetic variants (genes) or combinations of genetic variants which predispose the individual to such an inflammatory disease; the presence of physical findings, laboratory test results, imaging findings, marker test results (also termed “biomarker” test results) associated with development of the inflammatory disease, or marker test results associated with development of a metabolic disease; the presence of clinical signs related to the inflammatory disease; the presence of certain symptoms related to the inflammatory disease (although the individual is frequently asymptomatic); the presence of markers (also termed “biomarkers”) of inflammation; and other findings that indicate an individual has an increased likelihood over the course of their lifetime to develop an inflammatory disease or disease associated with inflammation. Most individuals at increased risk for development of an inflammatory disease or disease associated with inflammation are asymptomatic, and are not experiencing any symptoms related to the disease that they are at an increased risk for developing.

### Immunogenic dsRNA Score (IDS) Determination

Immunogenic dsRNAs act in aggregate to elicit cellular immunogenicity. Risk variants of these immune-related diseases show directional effects, where across multiple autoimmune and immune-related diseases, there is an overwhelmingly negative direction of effects, i.e., risk GWAS variants lead to less dsRNA editing in general. The directional effects of RNA editing are even more significant when tested in tissue types of disease relevance. Associations are shown for autoimmune diseases including, for example, autoimmune thyroid disease, celiac disease, inflammatory bowel disease, primary biliary cirrhosis, systemic lupus erythematosus, multiple sclerosis, psoriasis, type 1 diabetes (T1D), rheumatoid arthritis; for atopic conditions such as asthma and atopic dermatitis; and for other conditions with a significant inflammatory component, e.g. amyotrophic lateral sclerosis (ALS), coronary artery disease, Parkinson's disease, systemic sclerosis, Alzheimer's disease, and levels of high and low density lipoproteins and triglycerides.

The IDS is correlated with immune-related disease, and which can be predictive of response to therapy, e.g. therapy directed at reducing the clinical sequelae of undesirable activity in dsRNA sensing pathways. The IDS can also be used in the stratification of individuals for clinical trials, and to identify individuals responsive to therapy that reduces the clinical sequelae of undesirable activity in dsRNA sensing pathways. Such undesirable activity may include, without limitation, decreased ADAR-mediated RNA editing systemically or in targeted tissues or cell types; increased MDA5 activity systemically or in targeted tissues or cell types; and increased type I interferon expression systemically or in targeted tissues or cell types. These activities provide points of therapeutic intervention for individuals determined to be at risk. In some embodiments the individual has been previously diagnosed with an immune-related disease of interest.

In some embodiments a method of determining an individual's immunogenic dsRNA score (IDS) comprises genotyping said subject at a plurality of risk alleles associated with undesirable activity in dsRNA sensing pathways, and generating a double-stranded RNA (dsRNA) burden score by weighting the number of risk alleles that decrease RNA editing levels by (a) the effect size of association with the disease, and (b) the effect size of their association with RNA editing level. The risk alleles may be selected from SNPs listed in Supplementary Table 3 of Li et al. (2022) Nature 608:569-577. In some embodiments the immunogenic dsRNA score determination further comprises genotyping the individual at one or more alleles associated with RNA editing level changes.

In some embodiments the immunogenic dsRNA score further comprises determining the specific dsRNAs of which sufficient RNA editing levels are required to maintain self-tolerance and avoid MDA5 activation (termed as immunogenic dsRNAs). The immunogenic dsRNAs may be selected from edited genomic regions listed in Supplementary Table 3 of Li et al. (2022) Nature 608:569-577. For example, as disclosed in Example 1 of the present specification, SNPs can be computationally colocalized at GWAS loci of diseases of interest associated with immune responses, to provide a set of immunogenic dsRNA candidates.

Alternatively, immunogenic dsRNA can be experimentally derived in a cell or tissue of interest by de novo identification of editing clusters, which are defined as (1) it has at least five editing sites and (2) any two adjacent sites are less than 120 nt apart. Identifying editing clusters allows grouping all sites in a dsRNA to quantify the overall editing level, defined as the cluster editing index. A comparative analysis searches for editing clusters whose editing index is significantly higher in ADAR1 p150—than ADAR1 p110-complemented cells. Editing clusters that are similarly edited by p150 and p110 have a low potential for immunogenicity. Clusters preferably edited by p150 are immunogenic dsRNA candidates. In some embodiments the immunogenic dsRNAs determination further comprises dsRNAs listed in Table S1 and Table S3 of Sun et al., bioRxiv 2022.08.29.505707, “A Small Subset of Cytosolic dsRNAs Must Be Edited by ADAR1 to Evade MDA5-Mediated Autoimmunity”, see also Example 2.

In some embodiments the immunogenic dsRNA score further comprises determining the tissues or cells of disease relevance and the expression level of immunogenic dsRNAs derived therefrom. The tissues or cells of disease relevance can be selected by (a) measuring interferon (IFN) score by aggregated expression levels of interferon-stimulated genes (ISGs) to determine the highest tissue or cell (ISGs may be selected from a list of 331 genes in Table 2), and (b) measuring the aggregated expression level of immunogenic dsRNAs in disease tissue scRNA-seq data using the single-cell disease relevance score (scDRS) method described in Zhang et al. (2022) Nature Genetics 54:1572-1580. The expression level of dsRNA can be further weighted to account for the immunogenicity of dsRNAs. The weights are determined by the genetic effects of dsRNAs in increasing disease risk as measured in GWAS of inflammatory diseases. In the future when it is feasible to experimentally determine the potency of dsRNA in triggering MDA5 activation, this information can also be used as weight.

In some embodiments the immunogenic dsRNA score further comprises inputting additional clinical indicia, including without limitation one or more of: age, gender, family history, age of onset of said disease, duration of said disease, and the like.

The immunogenic dsRNA score (IDS) can be calculated based on factors as set forth above. For a given individual, a predicted IDS of the disease relevant tissue/cell-type higher than the IDS thresholds for a given disease is considered as indicative of high risk. The IDS threshold can be experimentally determined by performing MDA5 knockout assays in human donor-derived disease relevant cell lines, which include both patients and healthy controls. Cellular IFN response level is used as the readout of MDA5 knockout. For cells with significantly reduced IFN response upon MDA5 knockout, the corresponding donors should be considered as of high risk. Conversely, for cells without significantly altered IFN response upon MDA5 knockout, the corresponding donors should be considered as of low risk. It should be noted that high/low risk is a relative term in the context of MDA5-mediated disease risk. Once the IDS threshold is determined and validated using a sufficient number of human donor-derived cell lines, further experiments will not be needed for individual-level prediction in the future. The determination and validation of the IDS threshold need to be performed for each different inflammatory disease. In some embodiments the IDS (Y) is determined by the equation:

$${Ψ}^{k} = {\sum }_{i = 1 , j = 1}^{n} {β}_{ij}^{k} \times {ϕ}_{ij}^{k} \times {ε}_{i}^{k}$$

where

$${β}_{j}^{k}$$

is the effect size of j^{th} SNP for the disease in k^{th} tissue/cell type,

$${ϕ}_{ij}^{k}$$

is the effect size of i^{th} dsRNA with j^{th} SNP for RNA editing level in k^{th} tissue/cell type and

$${ε}_{ij}^{k}$$

is the expression level of i^{th} dsRNA in k^{th} tissue/cell type, and optionally further comprises clinical indicia as described above.

Genotyping can be performed on a DNA sample from the individual for single-nucleotide polymorphisms, e.g. by sequencing, hybridization, etc. as known in the art. The methods disclosed herein identify a subset of SNP loci, set forth in Supplementary Table 3 of Li et al. (2022) Nature 608:569-577, that predict immunogenic dsRNA levels by quantitative trait loci (QTL) mapping in a plurality of individuals. DNA sequencing is used to classify the SNP loci that have a p-value score below a pre-set threshold, with a directional effect that increases immunogenic dsRNA level. Training on genome-wide association study (GWAS) data is used to assess distribution of IDS between cases and controls for a given autoimmune/inflammatory disease, and determine the IDS thresholds to generate predictions for high risk for the disease. The methods may include genotyping of at least about 100, at least 200, at least about 500, at least about 1000, at least about 5000, at least about 10,000, at least about 15,000, at least about 20,000, at least about 25,000, at least about 30,000 SNPs, and may genotype substantially all of the SNPs listed in Supplementary Table 3 of Li et al. (2022) Nature 608:569-577.

### Treatment

In some embodiments, an IDS is used to predict response to therapy, e.g. therapy directed at reducing the clinical sequelae of undesirable activity in dsRNA sensing pathways, where an IDS score indicative of higher risk indicates a predicted response to therapy directed at reducing the clinical sequelae of undesirable activity in dsRNA sensing pathways. Such undesirable activity may include, without limitation, decreased ADAR-mediated RNA editing systemically or in targeted tissues; increased MDA5 activity systemically or in targeted tissues; and increased type I interferon expression systemically or in targeted tissues. These activities provide points of therapeutic intervention for individuals determined to be at risk.

In some embodiments, an IDS score is used to stratify individuals for clinical trials directed at reducing the clinical sequelae of undesirable activity in dsRNA sensing pathways, and to validate clinical trial results. Patient stratification, also known as patient segmentation or patient profiling, is a process used in clinical trials to group patients into distinct subpopulations based on specific characteristics or criteria. The goal of patient stratification is to identify homogeneous groups of patients who are likely to respond similarly to a particular treatment or intervention being tested in a clinical trial. By stratifying patients, researchers can enhance the efficiency and effectiveness of clinical trials, leading to more accurate results and potentially accelerating the development of new therapies. Patient stratification helps to identify specific patient subgroups within a heterogeneous disease population, allowing researchers to better understand treatment efficacy and safety profiles for different patient profiles. Biomarkers such as an IDS score can be used to identify subgroups of patients who are more likely to benefit from a specific intervention, allowing for targeted enrollment in clinical trials. Patient stratification can be performed prospectively or retrospectively. Prospective stratification involves predefining the patient subgroups before initiating the clinical trial, based on prior knowledge or hypotheses. Retrospective stratification, on the other hand, involves analyzing patient data after the trial has started to identify subgroups based on observed responses. Various statistical methods are employed to identify and validate patient subgroups in clinical trials. These include clustering algorithms, classification models, regression analysis, and other machine learning techniques. The selection of appropriate statistical methods depends on the type and volume of data available for analysis.

An agent that inhibits MDA5 activity; an agent that inhibits type I interferon activity; an agent that increases ADAR1 activity or an agent that reduces immunogenic dsRNA formation or expression can be used as a therapeutic agent to reduce the clinical sequelae of undesirable activity in dsRNA sensing pathways. In some embodiments an agent is an inhibitor of MDA5, e.g. a small molecule inhibitor, an anti-sense RNA or RNAi agent, and the like. Agents that increase ADAR1 activity may include small molecules and coding constructs that increase expression in targeted tissues.

Dosage and frequency may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

An active agent can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified though various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

The active agents of the invention and/or the compounds administered therewith are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by oral administration. The active agents and/or other compounds may be systemic after administration or may be localized by virtue of the formulation, or by the use of an implant that acts to retain the active dose at the site of implantation.

Depending on the patient and condition being treated and on the administration route, the active agent may be administered in dosages of 0.01 mg to 500 mg/kg body weight per day, e.g. about 20 mg/day for an average person. Dosages will be appropriately adjusted for pediatric formulation.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

### Computer Methods

The methods disclosed herein include steps of data analysis, which may be provided as a program of instructions executable by computer and performed by means of software components loaded into the computer. Such methods include one or more of inputting genotyping data, e.g. sequence information for SNP loci of interest; inputting effect size of an SNP for disease association, inputting effect size of an SNP for RNA editing level association, inputting expression levels of dsRNA in tissues of interest. The methods can also include determination of threshold levels for a disease by training on GWAS data is used to assess distribution of IDS between cases and controls for a given disease, and determining the IDS thresholds to generate predictions for high risk for the disease. The methods can include solving the equation

$${Ψ}^{k} = {\sum }_{i = 1 , j = 1}^{n} {β}_{j}^{k} \times {ϕ}_{ij}^{k} \times {ε}_{i}^{k}$$

for these inputs. Other bioinformatics methods are provided for determining and quantitating when the IDS is physiologically relevant. The method may further comprise providing a computer-generated report comprising the analysis and determination of an IDS.

A computer system includes a central processing unit (CPU, also “processor” and “computer processor” herein), which can be a single core or multi core processor, or a plurality of processors for parallel processing. The system also includes memory (e.g., random-access memory, read-only memory, flash memory), electronic storage unit (e.g., hard disk), communications interface (e.g., network adapter) for communicating with one or more other systems, and peripheral devices, such as cache, other memory, data storage and/or electronic display adapters. The memory, storage unit, interface and peripheral devices are in communication with the CPU through a communications bus, such as a motherboard. The storage unit can be a data storage unit (or data repository) for storing data. The system is operatively coupled to a computer network with the aid of the communications interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network in some cases, with the aid of the system, can implement a peer-to-peer network, which may enable devices coupled to the system to behave as a client or a server.

The system is in communication with a processing system. The processing system can be configured to implement the methods disclosed herein. In some examples, the processing system is a nucleic acid sequencing system, such as, for example, a next generation sequencing system (e.g., Illumina sequencer, Ion Torrent sequencer, Pacific Biosciences sequencer). The processing system can be in communication with the system through the network, or by direct (e.g., wired, wireless) connection. The processing system can be configured for analysis, such as IDS analysis.

Methods as described herein can be implemented by way of machine (or computer processor) executable code (or software) stored on an electronic storage location of the system, such as, for example, on the memory or electronic storage unit. During use, the code can be executed by the processor. In some examples, the code can be retrieved from the storage unit and stored on the memory for ready access by the processor. In some situations, the electronic storage unit can be precluded, and machine-executable instructions are stored on memory.

The computer-implemented system may comprise (a) a digital processing device comprising an operating system configured to perform executable instructions and a memory device; and (b) a computer program including instructions executable by the digital processing device, the computer program comprising (i) a first software module configured to receive data pertaining to DNA sequencing; (ii) a second software module configured to relate the sequencing data to generate an IDS; and (iii) a third software module configured to calculate relative risk.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### RNA Editing Plays a Primary Role in Common Autoimmune and Immune-Related Diseases

A major challenge in human genetics is to identify the molecular mechanisms of trait- and disease-associated variants. To achieve this, quantitative trait loci (QTL) mapping of genetic variants with intermediate molecular phenotypes such as gene expression and splicing have been widely adopted. However, despite successes, the molecular basis for a considerable fraction of trait- and disease-associated variants remains unclear. Here we show that ADAR-mediated adenosine-to-inosine (A-to-I) RNA editing, a post-transcriptional event vital for suppressing cellular double-stranded RNA (dsRNA)-mediated innate immune interferon responses, is a primary mechanism underlying genetic variants associated with common autoimmune and immune-related diseases. We identified and characterized 30,319 cis RNA editing QTLs (edQTLs) across 49 human tissues. These edQTLs were significantly enriched in GWAS signals for autoimmune and immune-mediated diseases, exceeding the impacts of expression and splicing QTLs. Colocalization analysis of edQTLs with disease risk loci further pinpointed key, putatively immunogenic dsRNAs. Furthermore, inflammatory disease risk variants, in aggregate, were associated with reduced editing of nearby dsRNAs and induced interferon responses in inflammatory diseases. This unique directional effect fully agrees with the established mechanism that lack of RNA editing by ADAR1 leads to the specific activation of the dsRNA sensor MDA5 and subsequent interferon responses and inflammation. Our findings indicate dsRNA editing and sensing as a previously unappreciated mechanism underlying genetic risk of common inflammatory diseases, and present an actionable therapeutic approach to treating inflammatory disease patients by MDA5 antagonism.

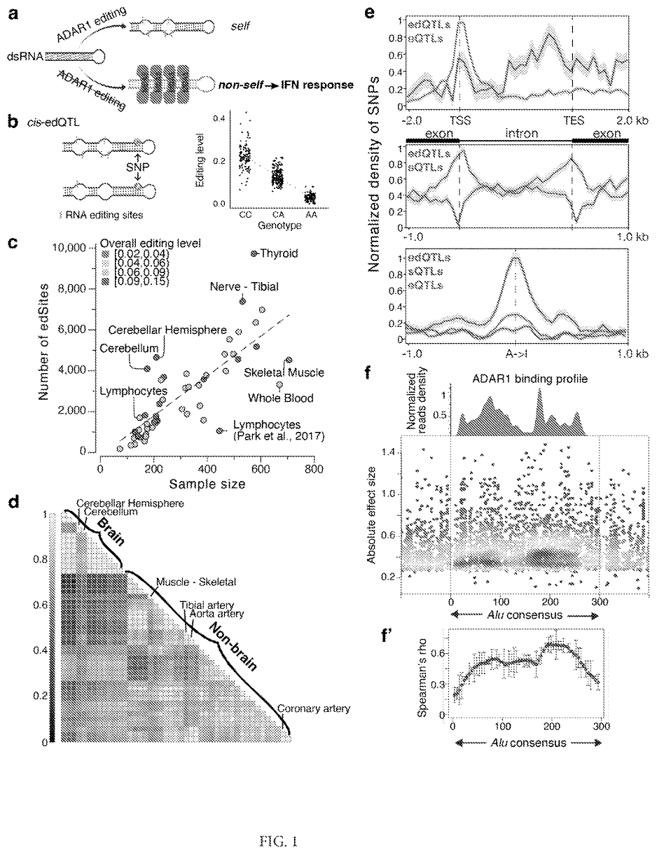

Identification of cis RNA editing QTLs in human tissues. In this study, we aimed to obtain a systematic understanding of the roles of A-to-I RNA editing in common and complex traits and diseases. We mapped cis editing QTLs (cis-edQTLs) using the GTEx V8 RNA-seq and genotype data (FIG. 1b; Methods), with substantial improvements over previous efforts, such as larger sample size and more comprehensive RNA editing site annotation. We first measured RNA editing levels-defined as the fraction of edited (‘G’) transcripts over total (‘A’ and ‘G’) transcripts—at single nucleotides (“sites”) across a catalogue of >2.8 million sites and obtained reliable editing level quantification for 14,993-60,581 sites per tissue type (FIG. 8a-b; Methods). To identify and control for potential confounding factors of editing level measurement, we performed principal component analysis (PCA) and found expression level of ADAR1 correlated with the top PC explaining 8% of overall editing level variance (FIG. 8c-d; Methods). Interestingly, moderately (40-60%) edited sites showed high levels of variation across individuals while lowly (<10%) or highly (>90%) edited sites had little variation (FIG. 8e). Similar observations were also made for DNA methylation level and RNA splicing ratios, in which a competition model between splicing isoforms has been proposed to explain how splicing ratios responded to local genetic perturbation.

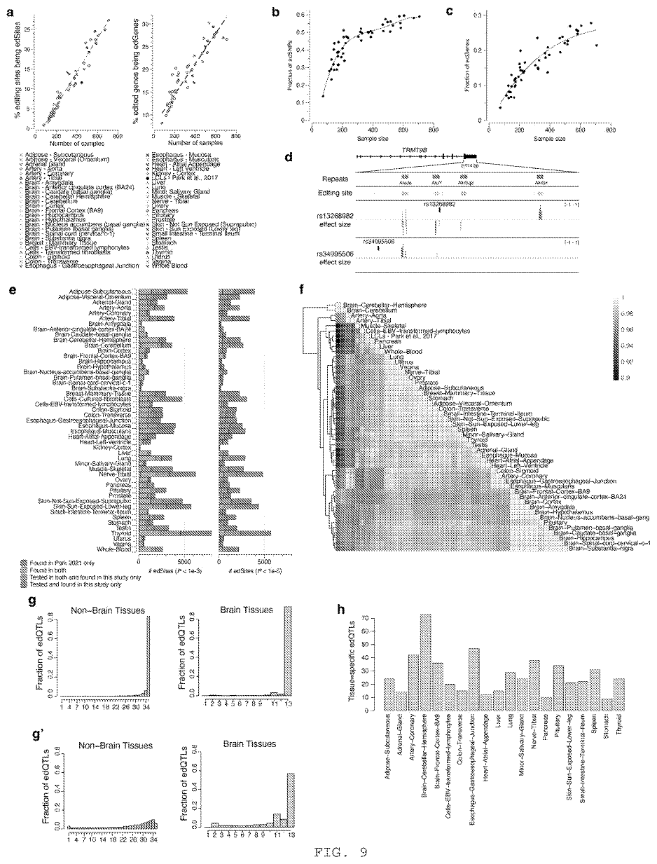

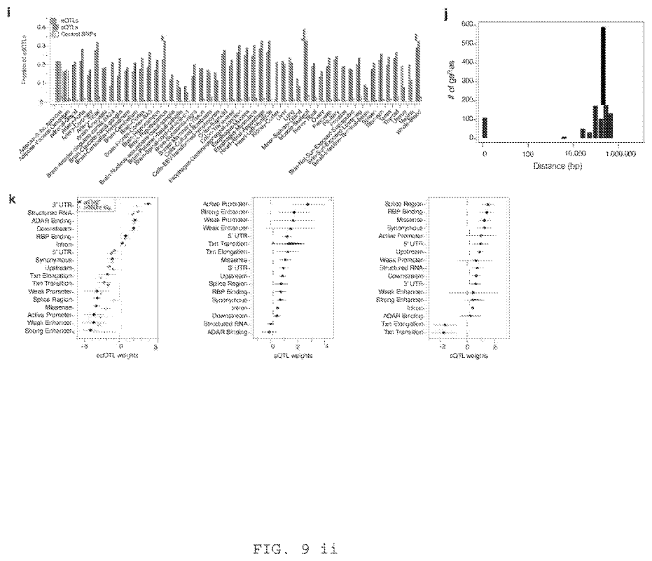

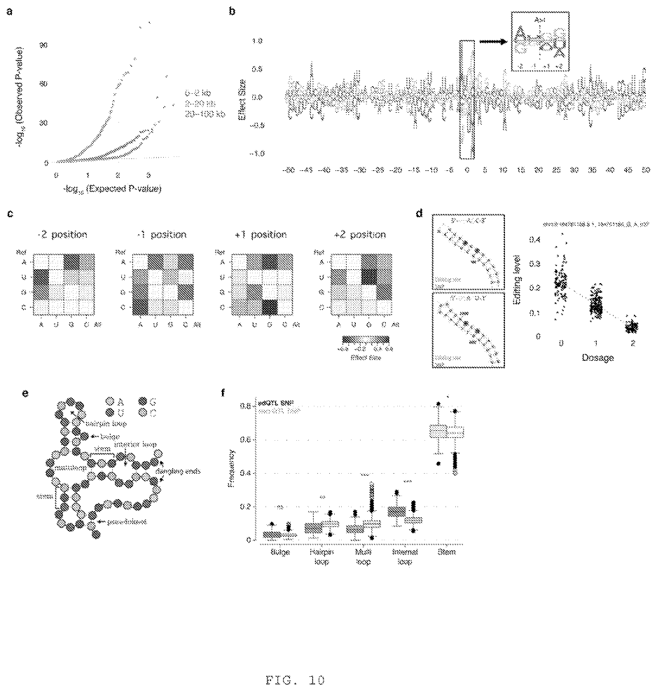

Next, we identified cis-edQTLs (hereafter denoted as edQTLs) in each GTEx tissue type using the QTL mapping pipeline widely adopted by the GTEx consortium. We considered proximal (within +/−100 kb of editing sites) common variants (MAF >5%) and editing sites observed in at least 60 samples of each tissue type (Methods). Of all 287,965 editing sites tested, we identified edQTL for 30,319 sites (10.6%, FDR<5%, permutation-based; Methods); hereafter we denote editing sites with edQTLs as edSites. In addition, edQTLs were present in 32% (7,165) of all edited genes, which we denote hereafter as edGenes (FIG. 9a). Within edGenes, the majority (~60%) of the edQTLs affected multiple editing sites at the same time (FIG. 9b), which is expected since editing sites tend to be located in close proximity. In addition, nearly 30% of the edGenes had multiple independent edQTLs (FIG. 9c), indicating that editing sites can be co-regulated by multiple independent SNPs (FIG. 9d). The number of edSites per tissue type was mostly dependent on sample sizes and overall editing levels (defined as transcriptome-wide fraction of edited transcripts over total transcripts) (FIG. 1c). Compared to a recent study using a smaller dataset, we identified ~9 times more edSites (FIG. 9e).

Characterization of cis RNA editing QTLs in human tissues. We compared the effects of edQTLs between tissues by performing a meta-analysis (Methods). We observed that cis genetic effects on RNA editing were highly consistent across tissues (FIG. 1d; FIG. 9f-g′). More specifically, the direction of effects across all tissues was consistent for 87.6% of the cis-edQTLs (FIG. 1d; FIG. 9f), while only 538 edQTLs showed tissue-specific effects (>2-fold difference in the magnitude of effects, Methods) (FIG. 9h). In addition, 31.1% of edQTLs were found only in one tissue due to tissue-specific gene expression. Our results indicate that the genetic landscape of RNA editing in human is complex and requires multi-tissue data to fully describe.

We next compared edQTLs with expression QTLs (eQTLs) and splicing QTLs (sQTLs) identified in the same GTEx dataset. Unlike eQTLs that were enriched near the transcription start sites (TSSs), edQTLs were enriched in the 3′-UTRs, which was reflective of the large proportion (43%) of edSites located in the 3′-UTRs (FIG. 1e, top panel). Consistent with previous report, sQTLs, but not edQTLs, were enriched near splicing junctions (FIG. 1e, middle panel). As expected, edQTLs were strongly enriched near editing sites (FIG. 1e, bottom panel). The subtle enrichment observed for eQTLs and sQTLs near editing sites suggests potential overlaps between edQTLs and eQTLs or sQTLs. Indeed, 18.7% and 21.5% of edQTLs were also eQTLs or sQTLs, respectively (FIG. 9i; Methods), which agrees with a recent study of a smaller scale. In genes with both an edQTL and an eQTL, most of the lead SNPs for edQTL and eQTL were >10 kb away from each other and enriched in genomic elements of distinct functional annotations (FIG. 9j-k). Our findings highlight that most edQTLs are not detected as significant eQTLs or sQTLs in the GTEx dataset and potentially represent independent genetic regulatory effects.

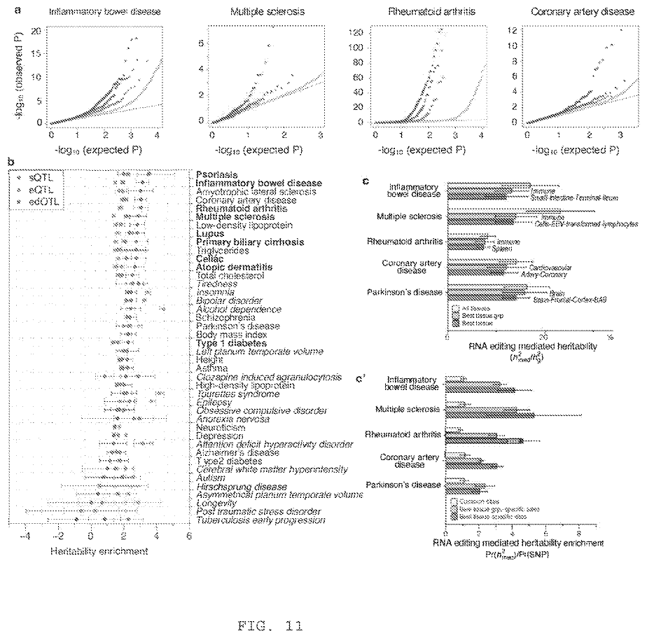

Our edQTLs map also provides a unique opportunity to investigate how RNA sequence and/or structural changes affect editing level in human tissues. We observed that edQTLs in closer proximity to the editing sites generally showed higher statistical significance (FIG. 10a), highlighting the effects of proximal regulatory elements such as ADAR-binding sites, RNA sequence motifs and RNA secondary structures. We subsequently conducted a focused meta-analysis on Alu elements where >99% of human editing sites reside due to ADAR binding preference (FIG. 1f; Methods). We observed that the effect size of edQTLs was significantly correlated with the estimated ADAR1 binding strength (FIG. 1f′), suggesting genetic variants could alter editing levels through affecting ADAR1 binding on RNAs. We then evaluated how changes in RNA sequences caused by genetic variants may affect the editing levels of nearby sites (Methods). We found that “AUAGG” sequence motif centering at the edited “A” was preferential for high editing levels (FIG. 10b-d), which agreed with previously reported “UAG” motif preferred by ADAR41. Furthermore, we observed that edQTL SNPs were 13-54% more enriched in RNA secondary structures recognized by ADAR, as compared to nearby non-edQTL SNPs (FIG. 10e-f).

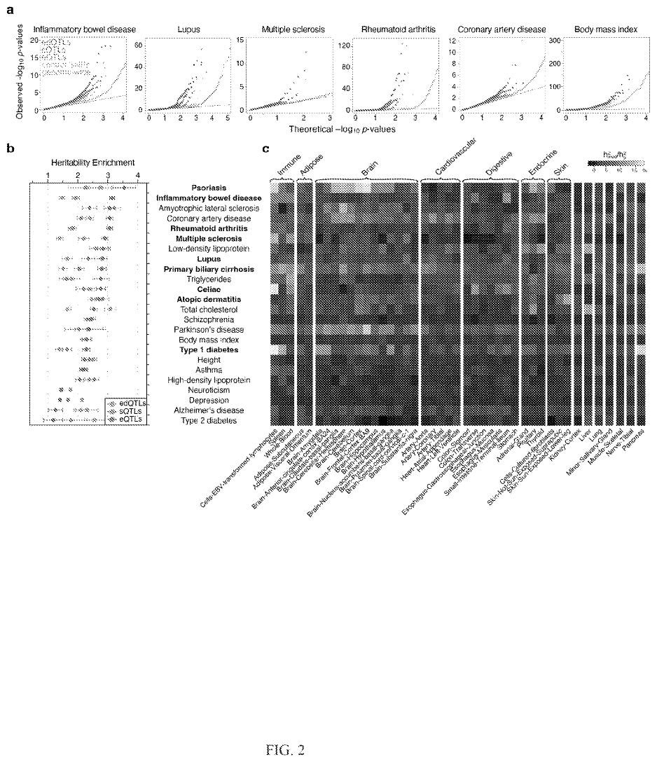

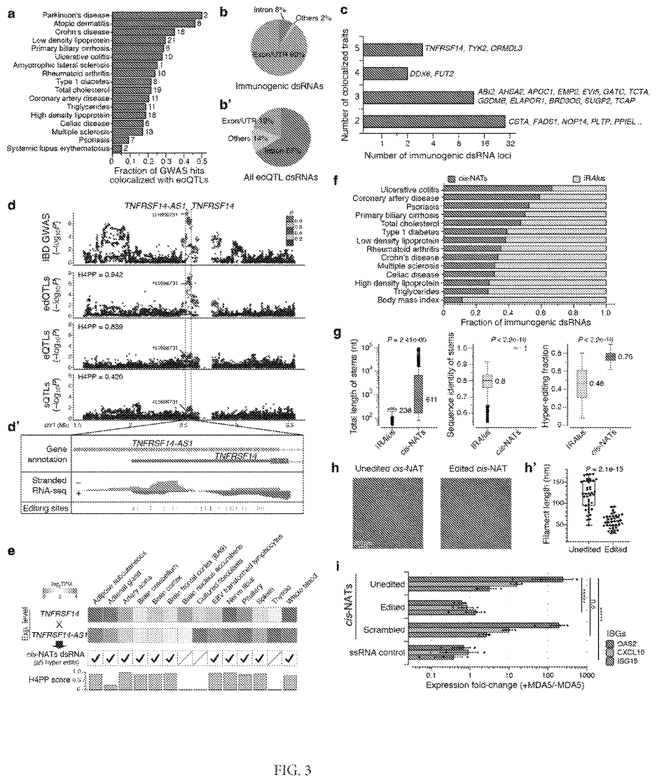

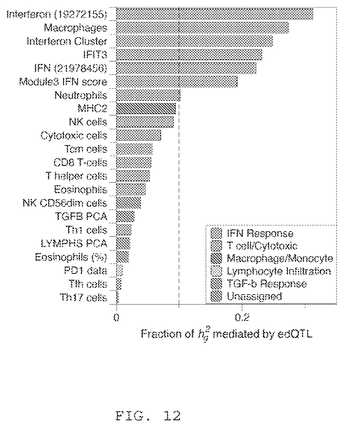

Genetic regulation of RNA editing contributes to disease heritability. To evaluate the potential role of A-to-I dsRNA editing in common genetic diseases and traits, we assessed the enrichment of edQTLs in GWAS signals of multiple studies. Similar approaches have been successfully applied in eQTL and sQTL studies to reveal major contributions of gene expression and RNA splicing to complex diseases and traits. We found that edQTLs are highly enriched in GWAS signals for autoimmune (as exemplified by inflammatory bowel disease (IBD), lupus, multiple sclerosis (MS) and rheumatoid arthritis (RA)) and immune-related diseases (as exemplified by coronary artery disease (CAD)), more than the randomly drawn control SNPs that were matched with number of SNPs in linkage disequilibrium (LD), allele frequency and gene density (FIG. 2a; Methods). Although eQTLs and sQTLs were also enriched, consistent with previous studies, edQTLs had effects of larger magnitude than either eQTLs and sQTLs (FIG. 2a). This observation still held true when only considering eGenes and sGenes expressed comparably to edGenes, which are generally more highly expressed (FIG. 11a). We further confirmed these results by quantitatively assessing the enrichment of QTL explained heritability for GWAS of complex diseases and traits listed in Table 1. In 8 out of 9 autoimmune diseases tested, edQTLs were more enriched in heritability than eQTLs and sQTLs (FIG. 2b). In addition, edQTLs were enriched in amyotrophic lateral sclerosis, CAD, triglycerides and low-density lipoproteins (FIG. 11b), all of which are implicated with immune functions.

TABLE 1
Complex diseases and Traits

Trait/Phenotype + A1:A37
Alcohol Dependence
Alzheimers
Amyotrophic Lateral Sclerosis
Anorexia Nervosa
Asthma
Atopic Dermatitis
Attention Deficit Hyperactivity
Disorder
Autism Spectrum Disorder
Bipolar Disorder
Blood Lipids
BMI and Height
Celiac Disease
Cerebral White Matter
Hyperintensity
Clozapine-Induced Agranulocytosis
Coronary Artery Disease
Depression
Epilepsy
Hirschsprung Disease
Inflammatory Bowel Disease
Insomnia
Longevity
Multiple sclerosis
Neuroticism
Obsessive Compulsive Disorder
Parkinsons
Planum Temporale Volume
Posttraumatic Stress Disorder
Primary Biliary Cirrhosis
Psoriasis
Rheumatoid Arthritis
Schizophrenia
Systemic Lupus Erythematosus
Tiredness
Tourette's Syndrome
Tuberculosis Early Progression
Type 1 Diabetes
Type 2 Diabetes
Vitiligo

The multi-tissue GTEx data also allowed us to test the tissue-specific contribution of edQTLs in common diseases. Using a recently published approach that specifically aims to distinguish directional, mediated effects from nondirectional pleiotropic and linkage effects, we estimated the proportion of heritability mediated by edQTLs in 24 diseases and traits.

Again, an overall higher proportion of heritability was mediated by edQTLs (0.18±0.04) than by eQTLs (0.11±0.02, estimated using the same methods applied to GTEx V8) in autoimmune and immune-related diseases, but not in traits without implicated immune contribution (0.033±0.02). Notably, edQTLs of tissues of the immune system (lymphocytes, spleen and whole blood) collectively explained the largest proportion of heritability in most autoimmune and immune-related diseases tested (FIG. 2c; FIG. 11c). The edQTLs mediated heritability were also enriched in known tissues of disease relevance, such as digestive tissues (small intestine and colon) for IBD and Celiac disease, brain tissues for ALS and Parkinson's, cardiovascular tissues for CAD, and pancreas for T1D (FIG. 2c; FIG. 11c′).

In addition to the GWAS of autoimmune and immune-related diseases, we evaluated the edQTL enrichment in GWAS signals of 33 highly heritable immune traits defined in a recent study. We found that edQTLs were more enriched for GWAS of interferon response-related immune traits than other immune traits tested (FIG. 12). This is consistent with genetic data from human and mouse showing that lack of ADAR1 RNA editing triggers an MDA5-medited innate immune interferon. Together, our data provide compelling evidence that edQTLs make a significant contribution to the heritability of autoimmune and immune-related diseases, presumably through triggering the interferon response mediated by dsRNA editing and sensing.

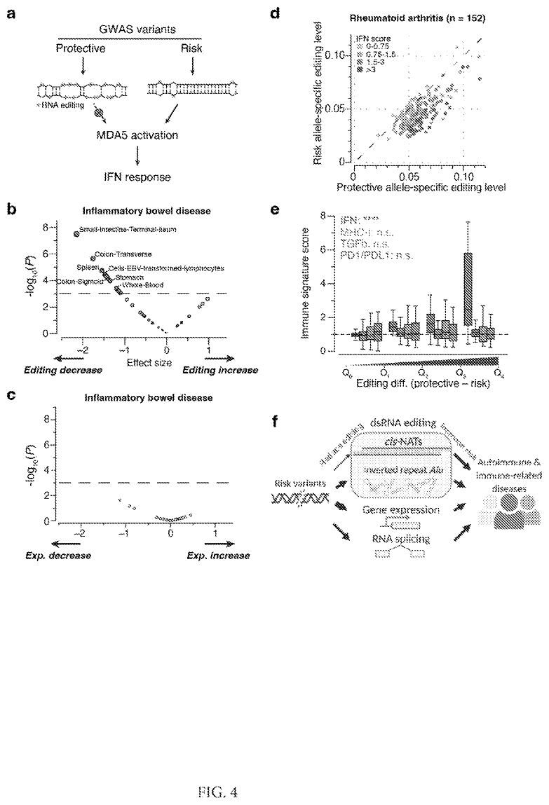

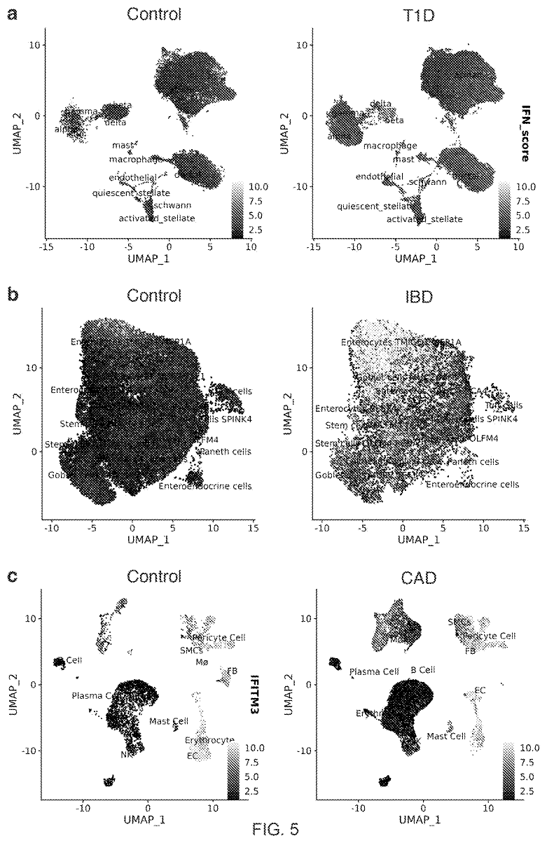

Identification and characterization of putatively immunogenic dsRNAs with disease relevance. The genome-wide significant enrichment of edQTLs in heritability of autoimmune and immune-related diseases prompted us to pinpoint specific dsRNA loci of disease relevance. We denote these dsRNAs as putatively immunogenic dsRNAs whose sufficient editing is important to suppress autoimmunity, presumably by evading MDA5 activation. We identified putatively immunogenic dsRNAs by systematically investigating signal colocalization between edQTLs found in 49 human tissues and previously reported genetic variants obtained from 24 GWAS studies, including 17 diseases and traits implicated with immune functions (Methods). In total, we identified 1,974 colocalization events (loci×edGenes), linking 17 immune-related diseases to 194 genes expressing putatively immunogenic dsRNAs (FIG. 3a). Because dsRNAs must be sensed by cytosolic MDA5 to elicit immunogenicity, we reasoned that they should be predominantly located in exons/UTRs instead of introns to be present in the cytosol. Indeed, of the 194 putatively immunogenic dsRNAs, 178 (92%) were located in exons, specifically in UTRs where long dsRNA structures are often formed. (FIG. 3b). In contrast, of all 15,620 dsRNAs associated with edQTLs identified in this work, only 2,967 (19%) were located in exons/UTRs, compared to 10,465 (67%) located in introns where most IRAlu dsRNAs reside (FIG. 3b′).

Next, we characterized the 194 putatively immunogenic dsRNAs. A majority of them (130, 67%) were located in IRAlus, which appeared to be substantially lower than expected because almost all long dsRNAs are thought to be formed by IRAlus in human (see below). Of the 194 dsRNA, 42 (22%) were shared between at least two diseases (FIG. 3c), suggesting that the immunogenicity of dsRNAs can serve as a common cause of susceptibility for multiple diseases. For example, a top candidate dsRNA found in TNFRSF14 was shared in five diseases/traits.

Validation of the putatively immunogenic dsRNAs. To experimentally validate the immunogenicity of dsRNAs, we first evaluated if MDA5 proteins could form filaments in vitro. We tested 3 dsRNA using negative stain electron microscopy (Methods). The dsRNA of each pair, but not their ssRNA controls, formed MDA5 filaments of varying lengths proportional to the dsRNA length (FIG. 3h;). Furthermore, the lengths of filaments were significantly reduced when the dsRNAs were in vitro edited by ADAR1 prior to MDA5 incubation (FIG. 3h-h′;), suggesting that immunogenic dsRNAs need to be edited to evade MDA5 sensing.

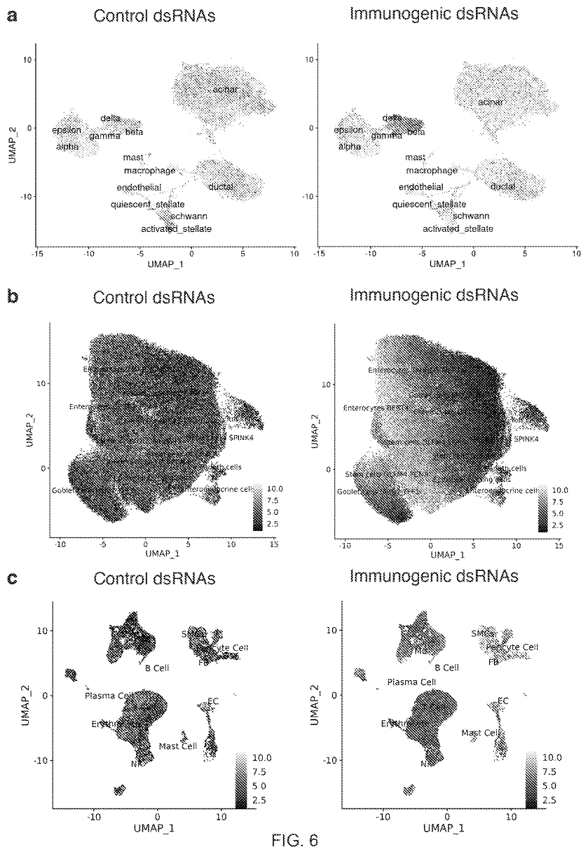

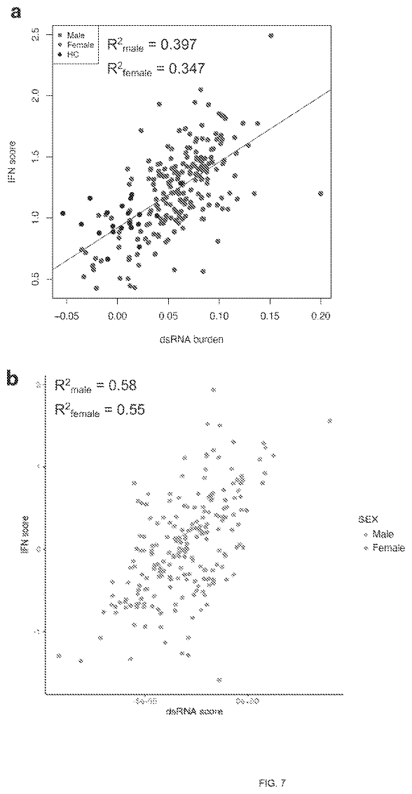

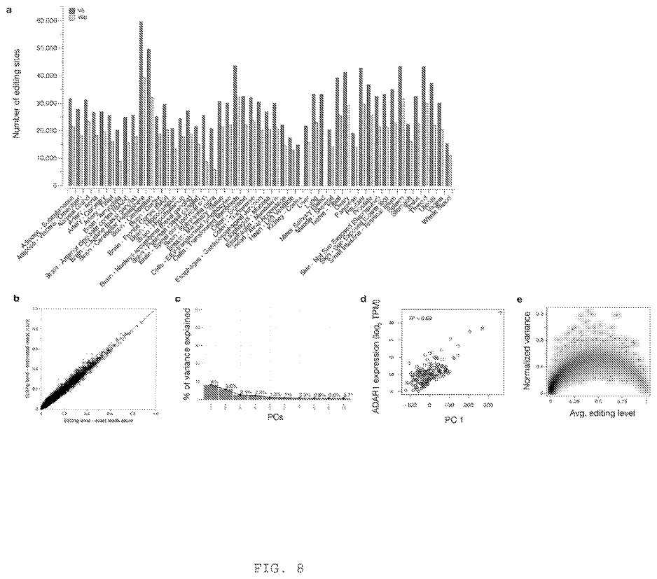

We next validated the immunogenicity of dsRNAs in human cells with multiple lines of evidence. First, we sought for evidence of dsRNA formation beyond the indicative hyper-editing. By using the publicly available structure mapping and ADAR1 binding data in human cells, we found that of 38 dsRNAs expressed in the corresponding cell lines, 29 showed RNA structure signals of high confidence (normalized icSHAPE score >0.7) for both strands in the overlapping regions, indicating formation of double-stranded structures between the sense and antisense transcripts (Extended Data FIG. 6e-f). Second, we evaluated the ability of dsRNAs to induce MDA5-dependent immunogenicity by over-expressing a candidate in human cells. We expressed the CTSA:PLTP pair, as a proof of concept, in ADAR1 editing-deficient cells with inducible MDA5 so that the exogenous dsRNAs would not be edited to diminish its immunogenicity (Methods). By transfection of a plasmid that expressed both sense and antisense transcripts at the overlapping region to mimic the dsRNA formation, we observed elevated immune response as indicated by the induction of three representative ISGs upon expression of MDA5 (FIG. 3i, Unedited vs. ssRNA control;). Third, we examined the immunogenicity of this dsRNA in cells with WT ADAR1. As expected, the immune response was significantly reduced as compared to ADAR1 editing-deficient cells (FIG. 3i, Unedited vs. Edited), suggesting that RNA editing by ADAR1 of the dsRNAs dampened their immunogenicity. Fourth, we hypothesized that the immunogenicity of dsRNA is dictated by the long dsRNA structure rather than the sequences. To test this, we generated a scrambled version of the dsRNA, with the overlapping sequences randomized while maintaining base-pairing complementary (Extended Data FIG. 7b). We found indistinguishable immune induction between the scrambled and WT version (FIG. 3i, Unedited vs. Scrambled), which validates our hypothesis. Taken together, our analyses indicate that the long dsRNA, without being edited by ADAR1, makes them highly potent ligands for MDA5 to trigger the innate immune responses, echoing their over-representation and importance in inflammatory diseases.

Reduced dsRNA editing underlies increased genetic risk of inflammatory diseases. Our analyses above suggest functional roles of the putatively immunogenic dsRNAs in autoimmune and immune-related diseases. In the well-established ADAR1-dsRNA-MDA5 mechanism, the activation of MDA5 and the subsequent immune responses is triggered by the lack of ADAR1-mediated editing of immunogenic dsRNAs. Very likely, these dsRNAs act in aggregate, not alone, to elicit the cellular immunogenicity. Therefore, we reasoned that risk variants of these aforementioned diseases should show directional effects to collectively reduce editing levels of the nearby dsRNAs. Reduced editing of dsRNAs would yield better ligands for the host dsRNA sensor MDA5, thus leading to elevated interferon response (FIG. 4a).

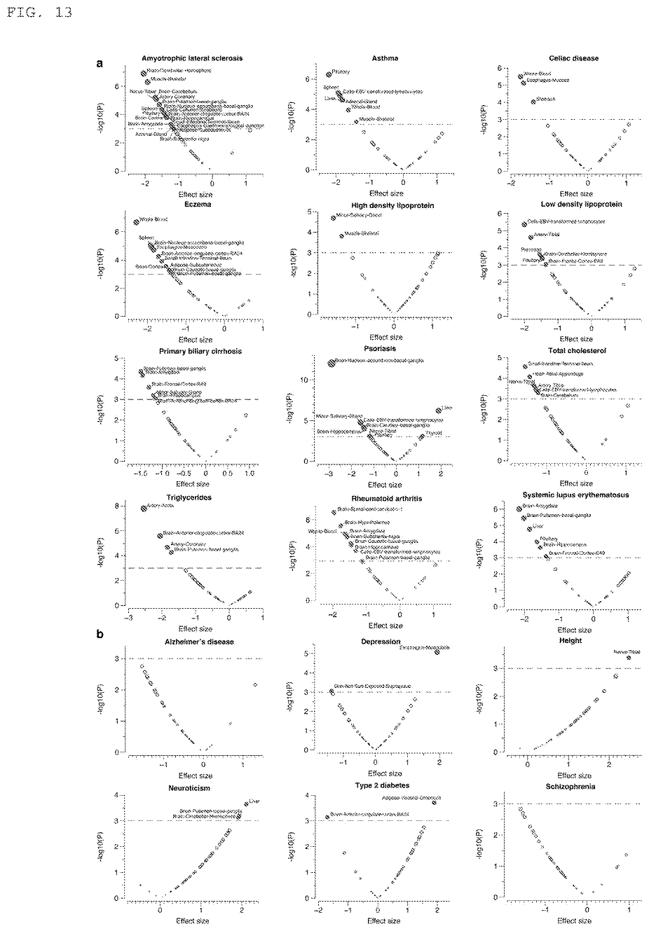

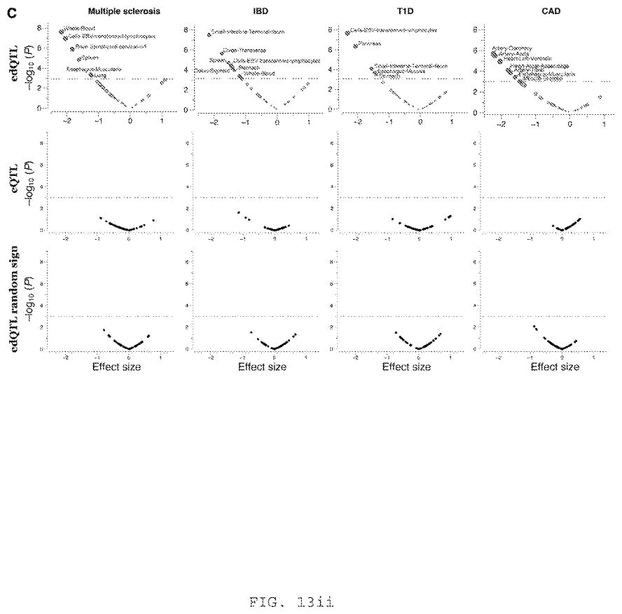

To test this model, we applied signed linkage disequilibrium profile (SLDP) regression to 24 complex traits and diseases (listed in Table 1) to assess the direction of genome-wide, collective effects of disease risk variants on editing levels while controlling for LD structure, allele frequency, gene expression and other potential systematic bias (Methods). Across multiple autoimmune and immune-related diseases, we detected overwhelmingly negative direction of effects (i.e., risk GWAS variants lead to less dsRNA editing in general), which supports our hypothesis that reduced editing levels of dsRNAs collectively lead to greater disease risk (FIG. 4b; FIG. 13). The directional effects of RNA editing were even more significant when tested in tissue types of disease relevance, as exemplified by digestive tissues for IBD, cardiovascular tissues for CAD, pancreas for T1D, and brain tissues for Parkinson's disease; while such directional effects were not observed for eQTLs or control non-directional SNPs (FIG. 4c; FIG. 13c).

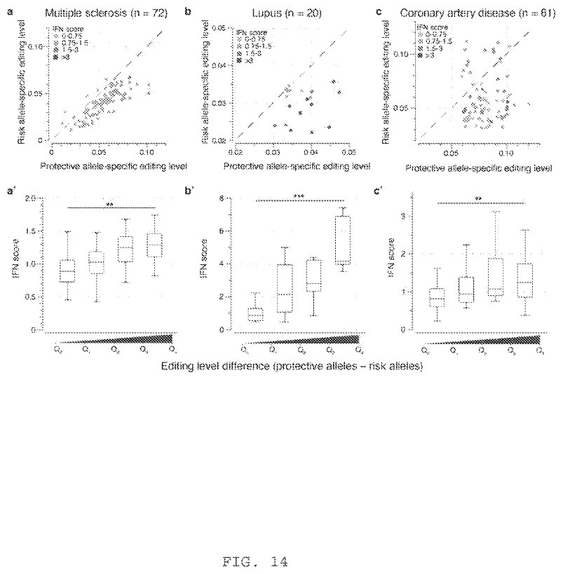

We further tested the directional effects using RNA-seq data from patient samples of four immune-related diseases. A challenge of such analysis is that the reduced editing of immunogenic dsRNAs leads to interferon responses, which may subsequently induce ADAR1 expression and the overall editing levels. Therefore, the initial reduction of editing driven the risk variants could be masked by the eventual increase of editing in a disease state. To overcome this, we examined allele-specific editing (ASED) levels, enabling the measurements of editing levels associated with risk vs. protective alleles. In total, we analyzed 152 synovial tissue samples from rheumatoid arthritis patients, 72 white matter samples from multiple sclerosis patients, 20 peripheral blood mononuclear cell (PBMC) samples from SLE patients and 81 coronary artery samples from CAD patients. For each disease cohort, we observed significantly reduced editing levels in association with risk alleles than protective alleles (FIG. 4d, relative reduction of 33.7±15.6%, paired t-test, p<2.7×10⁻¹²; FIG. 14). Moreover, the extent of editing level reduction was positively correlated with elevated IFN response as measured by IFN scores (Methods), but not other immune signatures (FIG. 4e). This finding nicely agrees with the mechanism that reduced RNA editing by ADAR1 results in dsRNA-mediated IFN responses, providing strong evidence for causal effects of dsRNA editing underlying common autoimmune and immune-related diseases.

The main function of dsRNA editing by ADAR1 on cellular transcripts is to evade MDA5-mediated dsRNA sensing and autoimmunity. Mutations in ADAR1 and MDA5 can trigger strong dsRNA-mediated immune responses and lead to very rare autoimmune diseases. In this work, we aimed to understand how the editing status of dsRNAs may contribute to common human diseases. We found that common genetic variants associated with RNA editing levels were significantly enriched in GWAS signals of common autoimmune and immune-related diseases and accounted for a significant fraction of disease heritability. We further showed a directional effect that GWAS-associated genetic risk variants, in aggregate, generally reduce the editing levels of nearby dsRNAs, particularly in disease-relevant tissues. The less edited dsRNAs serve as better substrates of MDA5 to trigger interferon response, which is consistent with the well-established ADAR1-dsRNA-MDA5 axis (FIG. 4f). Our findings suggest that the enrichment of risk variants, by collectively reducing the editing levels of associated dsRNAs (likely in the number of hundreds), results in MDA5-dependent interferon response and inflammation. This is further supported by previous findings of MDA5 protective, loss-of-function alleles in GWAS studies for several inflammatory diseases. Taken together, our work, built on human genetics and well-established mechanisms, implicates an actionable therapeutic approach to treating inflammatory disease patients by antagonizing MDA5.

In summary, our work presents RNA editing as an important mechanism underlying numerous autoimmune and immune-related diseases, and sheds light on manipulating the dsRNA editing and sensing pathway for potential therapeutic developments.

### Methods

Editing level quantification in GTEx samples. The GTEx gene expression data used in this study was obtained from the GTEx portal (GTEx Analysis V8 release) and measured in Transcripts Per Million (TPM). The editing level was quantified on the GTEx Analysis Release V8 (dbGaP Accession: phs000424.v8.p2), which consists of a total of 17,382 RNA-seq samples, sequenced in 76 bp long paired-end reads.

We first compiled a list of reference editing sites for quantification. By incorporating known sites in RADAR database, tissue-specific sites identified in GTEx V6p, and recently published hyper editing sites, we finalized a list of 2,802,572 human editing sites. To quantify the editing levels, we computed the ratio of G reads divided by the sum of A and G reads at each site. We included 15,201 RNA-seq samples from 838 donors with matching genotypes in 49 tissues with sample size ≥70 for editing level quantification and downstream analyses. For duplicated reads tagged during the RNA-seq mapping process, we chose to retain the read with the highest base quality (if they were the same base quality, a random read was selected). We required that in each tissue, editing sites should be covered by >20 non-duplicated reads in >60 samples to be considered as testable in the downstream analyses, and the variation across samples is non-zero for edQTL mapping. After applying the above filters, we obtained 14,993-60,581 sites for downstream analyses, varying across tissues (FIG. 8a). Codes used for editing level quantification in GTEx V8 data and the reference editing site list are available at: https://hub.docker.com/r/vanessa/mpileup/.

cis-edQTL mapping. We first used principal components analysis (PCA) to identify potential confounders in editing level measurements. Editing level measurements are usually less confounded than gene expression. We found that the top 10 PCs collectively contributed to ~20% of total editing level variance (FIG. 8c) and PC1 was highly correlated with the expression level of ADAR1, agreeing with previous observation. The numbers of PCs regressed out from editing level measurements (see below) were chosen to maximize the number of detected cis-edQTLs with ≤5 PCs needed in all tissues (we tested 0 to 10 PCs).

To map edQTLs, we considered all SNPs with MAF >0.05 within ±100 kb of editing sites. Variant call files (VCF) of genotype data for 838 GTEx donors with matching RNA-seq data were obtained from dbGAP (accession ID: phs000424.v8) based on GRCh38/hg38 reference.

We used FastQTL for edQTL mapping. Raw editing level measurements were logit-transformed before regressed out PCs, and then normalized to N (0, 1) distribution across individuals within each tissue. Top 3 genotype PCs together with sex and age were used as covariates for edQTL mapping. For each editing site, the adaptive permutation mode was used with the setting—permute 1000 10000. The beta distribution-extrapolated empirical p-values from FastQTL were used to calculate trait-level q-values with a fixed p-value interval for the estimation of π₀ (lambda=0.85). A false discovery rate (FDR) threshold of ≤0.05 was applied to identify editing sites with at least one significant edQTL.

To identify the list of all significant variant-site associations for cis-edSites, a genome-wide empirical p-value threshold was defined as the empirical P-value of the site closest to the 0.05 FDR threshold. Nominal p-value threshold was then calculated for each editing site based on the beta distribution model (from FastQTL) of the minimum p-value distribution obtained from the permutations for the gene. For each editing site, variants with a nominal p-value below the threshold were considered significant and included in the final list of variant-site pairs.

We implemented in the edQTL mapping pipeline with strict removal of gene expression levels to control for potential confounding effects of gene expression. More specifically, for each editing site, we included the expression level of its host gene (measured in TPM values) as an additional covariate alongside genotype and phenotype covariates to be regressed out from the editing level measurements and used the residuals for edQTL mapping.

Tissue sharing of edQTLs. We applied the multivariate adaptive shrinkage implemented in MashR to compare edQTL effect size between tissues. To fit the MashR model, we used the set of ~4,000 edQTLs shared between 20 major tissue types to learn the MashR prior, and then fit the MashR model using 40,000 randomly selected variant-trait pairs for the same set of edSites.

We learned data-driven MashR priors by: 1) PCA with number of PCs=3; 2) empirical covariance of observed Z-scores. The data-driven covariances were further denoised by calling cov_ed in MashR. Furthermore, we included the set of canonical covariances as described in “cis-edQTL mapping” as an additional MashR prior. We fit the MashR model using the set of randomly selected variant-trait pairs with the error correlation estimated by applying estimate_null_correlation function in MashR and the priors obtained above. The resulting MashR model was used to compute the posterior mean, standard deviation, and local false sign rate (LFSR) for a given variant-trait pair. Effect size estimates and LFSR outputted by MashR were used as metrics of edQTL magnitude and activity respectively.

Comparative analyses between edQTLs, eQTLs and sQTLs. We obtained eQTLs and sQTLs mapped by the GTEx Analysis Working Group using GTEx V8 data from dbGaP (accession: phs000424.v8.p2). We assessed the sharing of edQTLs with eQTLs and sQTLs by applying Storey's TT1. More specifically, we identified significant SNP-editing pairs in a specific tissue, and then used the distribution of the P values for these pairs but tested for expression levels or splicing ratios to estimate TT1, the proportion of non-null associations.

For meta-gene analysis, we considered genes that have all three types of QTLs mapped in GTEx tissues. For each gene, lead SNP for each QTL was used to represent the corresponding cis signal (for edQTLs, lead SNP of each site was used and compared to lead SNPs of edQTLs and sQTLs of the same gene). We used the gene-level models based on the GENCODE68 V26 transcript annotation, where isoforms were collapsed to a single “transcript” per gene as reference to calculate distribution of QTL SNPs. All genes plus ±2 kb sequences were collapsed to a single meta-gene and further divided into 50 equal bins to calculate the density of SNPs. Splice junctions plus ±1 kb sequences and editing sites plus ±1 kb sequences were treated in the same way for density calculation.

ADAR1 CLIP-seq analysis. ADAR1 CLIP-seq was obtained from U87MG cells. Standard QC and filtering was performed using trim_galore (https://www.bioinformatics.babraham.ac.uk/projects/trim_galore/) to filter for high quality sequencing reads with adapter sequences removed. Reads shorter than 15 nt after trimming were discarded. To characterize the binding profile of ADAR1 in Alu repeats, all reads were first mapped to RefSeq genes using STAR with default settings. We then used BLASTN to align the mapped reads to Alu consensus sequences and only kept the best hit for each read (parameters: -evalue 1e-10-best_hit_score_edge 0.05-best_hit_overhang 0.25-perc_identity 50-strand plus). RNA-seq data of U87MG cells was used as a control set. We processed the control RNA-seq reads in the same way as CLIP-seq reads. Reads passing QC were mapped to U87MG reference genome sequence using STAR with default settings. The final mapped reads were then aligned to Alu consensus sequences as described above. To account for uneven reads coverage in Alu, we used the control RNA-seq to calculate the reads density level per base within the Alu consensus sequence and then computed the normalization factor per base by dividing the density level by the average density level of the entire Alu. Then for CLIP-seq data, read enrichment was calculated by multiplying the read count with the normalization factor.

RNA sequence motif. Given that ADAR recognizes the triplet “UAG” motif in a position-dependent manner, we sought to test if particular type(s) of nucleotide change at certain positions will have significantly stronger effect on alternating editing levels. For each edSite, we consider all significantly associated SNPs located within 50nt upstream and 50nt downstream of the editing site (100 positions in total). We compiled the ribonucleotide changes on RNA according to the SNP and the strand annotation of the gene (for example, if the SNP is a A-to-C mutation on the reverse strand, it will be interpreted as U-to-G on RNA). For each of the 100 positions, we assessed the average effects for all 12 types of ribonucleotide changes (FIG. 10c). Of note, not all symmetrical nucleotide changes show the same opposite effects. For example, C-to-A changes at −1 position have an average effect size of −0.8 while A-to-C changes at the same position have average effect size of +0.6. In some cases, the effects were in the same direction, such as A-to-U vs. U-to-A changes at +1 position (−0.3 vs −0.2) and G-to-U vs. U-to-G changes at +1 position (−0.4 vs −0.1). To simplify the signal and reduce the measurement noise, we further grouped the data to show the final effects of each of the 4 types trinucleotides, in regard to the alternative alleles. We plotted the sequence motif using ggseqlogo with the averaged effect size used to adjust for weight. Overall, a strong preference for A and U was observed at −2 and −1 positions, plus preference for Gs at +1 and +2 positions, showing a “AUAGG” motif.

RNA secondary structure. To understand how mutations affect editing levels through changes on RNA secondary structures, we predicted local RNA structures containing edSites and the associated SNPs. Since computational prediction of long RNA molecules is technically challenging, we limited the prediction window to +800 bp around each edSite (in total 1601 bp) and only considered the SNPs that fall into that window. We further restricted our analysis to the non-Alu editing sites. In total, we predicted secondary structures for 8,043 editing sites and subsequently annotated them with structural features using bpRNA (FIG. 10e-f). For each of the five structural features (pseudoknots and dangling ends were not considered due to insufficient data), we compared annotations of edVariants to SNPs that are not associated with editing levels but also found in the same local structure using Mann-Whitney U test.

Enrichment analysis of QTLs in GWAS signal. To make Q-Q plots of GWAS signal annotated with QTL information, we clumped the significant QTLs to obtain independent signals using PLINK (−-clump-r2 0.4—clump-kb 250). To control for overall higher gene expression levels of edGenes than eGenes and sGenes, we matched eGenes and sGenes to edGenes by the median expression levels across tissues. To generate a negative control set, we considered four features to match the control SNP set to edVariants: 1) MAF distribution. All edVariants were divided to 50 equal bins by allele frequency and the median MAF of each bin was used to select control SNPs of matching allele frequencies from the EUR set of 1000 Genomes; 2) the number of proxy SNPs in LD (LD “buddies”, r²=0.7). Similar to MAF filtering, LD buddies were sampled by matching with the median in each bin of edVariants distribution; 3) 3′-UTR density. edQTLs are strongly enriched around 3′-UTRs (average enrichment=2.1, compared to genome-wide), so we matched the number of 3′-UTRs in loci around the control SNPs (enrichment=2.0), using LD (r²>0.7) and physical distance (250 kb) to define the loci; 4) Distance to the nearest transcription termination site (TTS). We sampled the SNP-to-TTS distance (measured from the upstream side of TTS so that control SNPs are located within expressed region) to be within the same deviation estimated from the distribution of edQTL-to-TTS distance.

Heritability assessment and enrichment test in GWAS. We used the Mediated Expression Score Regression (MESC) pipeline for heritability analyses. We first estimated the overall editing scores from individual-level editing level quantified in each GTEx V8 tissue with matched genotype information. Five editing level PCs (described above in the “cis-edQTL mapping” section) were used as covariates. For meta-analysis across tissues, the editing scores were generated with edQTL effect sizes estimated using LASSO. Next, we estimated editing-mediated heritability (h²med) using the editing scores from both individual tissues and from tissue groups. GWAS summary statistic data of 24 traits described in Table 1 were obtained and converted to sumstats file format. LD scores computed from 1000 Genomes Phase 3 stratified over a modified version of the baselineLD model v2.0 were downloaded from the Broad institute. We kept the SNPs known to HapMap 3 as the SNP “universe”.

To test edQTL enrichment in immune-related traits, we used GWAS from Sayaman et al. In total, 33 immune traits were highly heritable among the 139 well-defined immune traits measured from ~9000 cancer patients enrolled in TCGA, and GWAS was subsequently performed on these 33 immune traits. The GWAS summary statistics data for these 33 immune traits, including the six IFN-response related immune traits, can be publicly accessed via: https://figshare.com/articles/dataset/Sayaman_et_al_TCGA_Germline-Immune_GWAS_Summary_Statistics/13077920.

Estimating directional effect of RNA editing on complex traits and diseases. We applied Signed LD profile (SLDP) regression method to estimate the directional effects as described in the original paper. We used the 1000 Genomes Phase 3 European genotypes to generate the reference panel for SLDP regression. LD scores of the same population were downloaded for reference panel https://data.broadinstitute.org/alkesgroup/SLDP/LDscore.tar.gz. We converted the GWAS summary statistic files as described above in the “Heritability assessment and enrichment test in GWAS” section. We processed the reference panel files by computing a truncated singular value decomposition (SVD) for each LD block in the reference panel, which were later used to weight the regression conducted by SLDP regression. The signed effect sizes of edQTLs were used as functional annotations to generate signed LD profiles. For the SNPs associated with multiple editing sites, we aggregated the effect sizes across the associated sites within the closest editing cluster using Stouffer's Z-score combination method. To explicitly control for the potential signed effects of gene expression, we also used the effect sizes of cis-eQTLs from the same set of genes called with edQTLs to generate a separate set of signed LD profiles to be used as a signed background model. As described in the original paper, directional effects of minor alleles in five equally sized MAF bins were included in the signed background model to control for systematic signed effects of minor alleles, which could arise from either population stratification or negative selection.

We obtained publicly available patient-derived disease samples for allele-specific editing (ASED) analysis. In total, we tested 152 synovial tissue samples from rheumatoid arthritis patients (GSE89408), 72 white matter samples from multiple sclerosis patients (GSE138614), 20 peripheral blood mononuclear cell (PBMC) samples from SLE patients (GSE122459) and 81 coronary artery samples from CAD patients (GTEx). For each sample, we phased the mapped RNA-seq reads nearby the corresponding disease GWAS risk variants, designating each read as belonging to either the risk or protective haplotype. We then quantified editing levels of the nearby editing sites (found on the same paired-end reads as the variants or its LD buddies, r²>0.4) for each haplotype and computed the overall editing level for sites near risk and protective alleles in each sample.

IFN scores were calculated with mRNA expression data (measured in TPM) normalized by median absolute deviation modified Z-score (ZMAD). The score was defined as the median ZMAD value of all signature genes in each sample. IFN signature genes were determined according to previous study in inflammatory disease.

GWAS download and preparation. We downloaded and consistently re-formatted public GWAS summary statistics from a variety of sources, using tools freely available at https://github.com/mikegloudemans/gwas-download. (“download” and “munge” modules)

SNP selection for colocalization analysis. Because the total number of GWAS traits×GWAS loci×QTL tissues×cis-QTL features is very large, it is computationally difficult and unnecessary to run every possible combination. We ran the “overlap” module given at https://github.com/mikegloudemans/gwas-download to generate a list of all trait/locus/tissue/feature combinations for which the lead GWAS SNP for that trait at the given locus has a p-value <5e-8, and overlaps an eQTL with p-value <1e-5 for the given QTL feature in the given tissue. Each of these combinations represented a single colocalization test to be performed. We performed this process for editing, splicing, and expression QTLs to generate a comprehensive list of 375,000 tests to run (26,000 edQTL; 183,000 sQTL 165,000 eQTL).

Colocalization analysis. For the set of tests determined in our previous step, we ran colocalization analysis using the tool COLOC with the default parameter settings, estimating allele frequencies from the full set of 1000 Genomes individuals. For each test, we obtained the H4 posterior probability (H4PP); that is, an estimate of the probability that the GWAS and QTL studies share a common causal variant. For subsequent analyses, we consider a test a “colocalization” if H4PP >0.9, unless otherwise stated. This threshold indicates a high level of support for colocalization. An implementation of the wrapper pipeline we used for performing COLOC analysis is available at https://github.com/mikegloudemans/ensemble-colocalization-pipeline.

For locuszoom plots shown in FIG. 3 and FIG. 4, we used the publicly available R package LocusCompareR to generate plots comparing the signal overlap between GWAS and edQTLs, sQTLs, and eQTLs in our locus of interest.

MDA5 and ADAR1 protein expression and purification. Human MDA5 protein (residues 298 to 1025) was expressed from pET-50b (+) in E. coli C41 cell, induced by adding 0.2 mM IPTG. After 20 h incubation at 18° C. with shaking, cells were harvested by centrifugation, resuspended in a buffer containing 20 mM Tris-HCl, 500 mM NaCl, 5% glycerol, 20 mM imidazole, 0.5 mM PMSF, pH 8.0, and lysed with high-pressure homogenization. The proteins were purified to homogeneity using Ni-NTA affinity, cation exchange, 2nd Ni-NTA affinity, size exclusion chromatography (in that order). HRV3C protease was added after 1st Ni-NTA affinity chromatography for tagged His6-NusA cleavage.

Human ADAR1p110 isoform was expressed in SF9 cells as recombinant protein with a N-terminal Twin-Strep-tag and purified by Strep-affinity chromatography. The cells were suspended and lysed in lysis buffer (20 mM Tris-HCl PH 8.0, 500M NaCl, 1 mM TCEP, 0.55% Triton-X100, 1 mM PMSF, protease inhibitors (Sangon Biotech) and 100 ng/mL RNase A) and purified by Strep affinity chromatography. The protein was eluted with 50 mM Tris-HCl, 200 mM KCl, 10% Glycerol, 1 mM TCEP, 2.5 mM D-Desthiobiotin.

Preparation of dsRNA and in vitro dsRNA editing. All dsRNAs were in vitro transcribed using T7 RNA polymerase. Two complementary strands were transcripted and purified separately. The pUC19 plasmids containing target sequences were linearized by EcoRI, extracted with phenol chloroform and precipitated with isopropanol. The in vitro transcription reaction was carried out at 37° C. for 4 h in 100 mM HEPES-K (pH 7.9), 10 mM MgCl2, 10 mM DTT, 6 mM NTP each, 2 mM Spermidine, 200 μg/mL linearized plasmid, 100 μg/mL T7 RNA polymerase. DNA template was digested with DNase I after reaction. Transcripts were purified by 8% denaturing urea PAGE, extracted from gel slices with 0.3M sodium acetate and precipitated with isopropanol. RNA of both complementary strands were mixed at molar ratio of 1:1 in annealing buffer (20 mM Tris-HCl PH 7.5, 50 mM NaCl, 1 mM EDTA) and heated to 90° C. for 3 min then slowly cooled to room temperature. For ADAR1 dsRNA editing in vitro, 0.64 ug annealed dsRNA was diluted and mixed with 0.4 nmol purified hADAR1-p110 to a total volume of 100 μL in buffer containing 50 mM Tris-HCl pH7.5, 60 mM KCl, 4% glycerol, 0.002% NP-40, 1 mM DTT, 1 mM EDTA, 1 mg/mL BSA and 0.4U/μL recombinant RNase inhibitor (Takara). Reaction was incubated at 37° C. for 60 min and edited RNA was purified using Absolutely RNA Nanoprep Kit (Agilent).

Negative staining electron microscopy. Samples including 0.38 μM HsMDA5 (298-1025) and 3.6 ng/μL dsRNA (regardless of length) were incubated on ice for 60 min with 1 mM ADP·AIF4 in buffer containing 20 mM HEPES, 100 mM NaCl, 2 mM MgCl2, 2 mM DTT, and pH 7.5. 5 μl of samples were applied to the glow-discharged 300 mesh carbon-coated copper grids (Beijing Zhongjingkeyi Technology), stained with 0.75% uranyl formate and air-dried. Data were collected on a Talos L120C transmission electron microscope equipped with a 4K×4K CETA CCD camera (FEI). Images were recorded at a nominal magnification of 45000×, corresponding to a pixel size of 3.17 Å per pixel. Filament lengths were measured using ImageJ.

Cell lines. HEK293T-ADAR1-E912A-iMDA5-mCherry-pIFN-Lucia cells were maintained in DMEM (Gibco, 11995-065) supplemented with 10% FBS (Gibco, 16140-071). This cell line was generated by transducing the HEK293T-ADAR1-E912A ADAR1-editing deficient cell line with three lentiviral vectors encoding the rtTA reverse transactivator, a doxycycline-inducible construct encoding MDA5 linked to mCherry, and the secreted luciferase Lucia (InvivoGen) under the control of an IFN-responsive promoter.

Plasmid construction. pK-mC3-CTSA-PLTP-mR3 was constructed using gene fragments synthesized by Twist Bioscience (San Francisco, California) and standard molecular cloning techniques. This plasmid contains an mClover3 cassette and an mRuby3 cassette facing towards each other with both under the control of separate EF-1a promoters and CAG enhancers. The 3′-UTR of CTSA has been attached to mClover3 and the 3′-UTR of PLTP has been attached to mRuby3 to mimic the endogenous overlapping state of these genes in the human genome. The scrambled version was built on the same backbone as the WT with the 208 bp overlapping region sequences randomized while maintaining complementarity between sense and antisense strands. pKER-mClover3 containing an EF-1a promoter- and CAG enhancer-driven mClover3 expression cassette with a rabbit beta-globin terminator was cloned to the same vector backbone and used as a ssRNA control.

Immunogenic dsRNA candidate overexpression assay. HEK293T-ADAR1-E912A-IMDA5-mCherry-pIFN-Lucia cells were plated on poly-L-lysine-coated (Millipore Sigma, A-005-C) 24-well plates (BD Falcon, 353047) at 100,000 cells per well. 24 hours later, cells were transfected with 500 ng per well of pK-mC3-CTSA-PLTP-mR3, pKER-mClover3, or Lipofectamine alone using Lipofectamine 3000 (Thermo-Fisher Scientific, L30001), according to the manufacturer's instructions. 24 hours post-transfection, media was aspirated and replaced with 500 μL of DMEM containing 0 or 0.1 μg/mL of Doxycycline. 24 hours post-Doxycycline addition, cells were washed with PBS and harvested with 0.5% trypsin. RNA was isolated from cells using the Monarch Total RNA miniprep kit (NEB, T2010S) cDNA was made from 500 ng of RNA using the iScript Advanced cDNA synthesis kit (Bio-Rad, 1725038). cDNA was diluted roughly two-fold with nuclease-free water and subjected to qPCR using Kapa SYBR FAST 2×qPCR Master Mix and primers derived from Primer Bank (https://pga.mgh.harvard.edu/primerbank/). 200 nM primers were used in each reaction and run on the Bio-Rad CFX96 following the manufacturer's instructions for Kapa SYBR Fast. Data were analyzed using the ΔΔCt method. Each assay was carried out as three separate biological replicates.

RT-PCR validation of plasmid expression. To determine if both cassettes on pK-mC3-CTSA-PLTP-mR3 were expressing, isolated RNA was subjected to Turbo DNase (Life Technologies, AM2238) treatment to digest any residual plasmid followed by purification with the Monarch RNA Cleanup Kit (NEB, T2040L) before being subjected to cDNA synthesis with iScript Advanced cDNA synthesis kit. cDNA was diluted roughly two-fold with nuclease-free water. 1 μL of the diluted cDNA was then amplified via PCR using OneTaq Quick-Load 2×Master Mix with Standard Buffer (NEB, M0486S) and primers designed to amplify from mClover3 through the CTSA 3′-UTR or from mRuby3 through the PLTP 3′-UTR. The resultant PCR products were subjected to electrophoresis on a 1% agarose gel.

### Example 2

Here, we developed novel genetic and computational approaches to identify immunogenic dsRNAs in different cell types. Using HEK293T cells and NPCs, we identified both shared and cell type unique immunogenic dsRNA candidates. In both cell types, we found that only a very small subset of dsRNAs are immunogenic without being edited, in contrast to previous findings. In addition to identifying Alu repeats, as expected, we discovered a novel class of dsRNAs that are formed by overlapping genes transcribed in opposite directions. We further characterized these immunogenic dsRNAs and identified features that distinguish them from non-immunogenic ones. Our work provides a framework to reveal immunogenic dsRNAs in the ADAR1-dsRNA-MDA5 axis, enabling future endeavors to identify key dsRNAs whose aberrant editing may lead to autoimmunity or immune-related diseases.

We developed a strategy to compare RNA editing at the whole dsRNA level. To identify dsRNAs in an unbiased fashion, rather than relying on annotations such as Alu repeats, we performed de novo identification of editing clusters as a proxy of long dsRNAs. To define an editing cluster, we required that (1) it has at least five editing sites and (2) any two adjacent sites are less than 120 nt apart. Using this novel approach, we identified 205,652 clusters in HEK293T cells, ~97% of which were in Alu and other repeats. The remaining ~3% of clusters in non-repeat regions were previously unannotated as dsRNA candidates. Identifying editing clusters allowed us to group all sites in a dsRNA to quantify the overall editing level, defined as the cluster editing index.

We performed a comparative analysis to search for editing clusters whose editing index is significantly higher in ADAR1 p150-than ADAR1 p110-complemented cells. We found that most (~96%) of the editing clusters were similarly edited by p150 and p110, suggesting that most of the dsRNAs have no or low immunogenicity. Of 39,079 testable editing clusters with sufficient coverage, we identified 420 (1.1%) clusters located in 316 genes as putative immunogenic dsRNAs, most of which were in the untranslated regions (UTRs) or intergenic regions but not in introns, consistent with the expectation that immunogenic dsRNAs recognized by cytosolic MDA5 would be embedded in processed RNAs exported to the cytoplasm. Of the clusters preferably edited by p150, some can be edited by p110 but to a lesser extent, while others are specifically edited by p150 only. In contrast, editing clusters more highly edited by p110 were largely present in introns, probably as a result of the overexpression of p110 in the nucleus. These intronic dsRNAs would not be able to be sensed by cytosolic MDA5 as they are removed during RNA splicing, thus rendering them non-immunogenic.

We made similar findings when we compared cluster editing indices between ADAR2- and ADAR2Δ^{NLS}-complemented cells. The lists of immunogenic dsRNAs identified from p150 vs. p110 or ADAR2 vs. ADAR2Δ^{NLS} comparisons were highly reproducible, particularly for immunogenic dsRNAs identified with higher statistical significance.

In addition, we compared the cluster editing indices between WT and ADAR1p150−/− cells to identify immunogenic dsRNAs that are significantly less edited in ADAR1p150−/− cells. We found that p150- and p110-preferred clusters were predominantly enriched in the cytoplasm and nucleus, respectively. These data confirm our findings that immunogenic dsRNAs are predominantly edited in the cytoplasm.

We examined if immunogenic dsRNA identified in this study were enriched in the RNase protection assay. We quantified the enrichment by the dsRNA expression fold-change in RNase-treated samples over untreated samples (in ADAR1 KO). We found that compared to control dsRNAs (randomly selected non-immunogenic dsRNAs located in 3′ UTR with comparable expression level to the immunogenic dsRNAs), the immunogenic dsRNAs were significantly more enriched for MDA5 binding.

Among the potentially immunogenic dsRNAs we identified, the vast majority were inverted repeats from the Alu family (90%) and other types of repetitive elements (8%). Although the intronic Alus comprise the vast majority of all Alus, they are almost excluded from the immunogenic group. The enrichment of immunogenic dsRNAs in mature mRNAs is fully expected because they would be sensed by MDA5 in the cytoplasm. Second, we quantitatively compared the contribution of different dsRNA features for immunogenic vs. non-immunogenic Alu repeats. We found no significant difference in the length of Alus, the base-pairing percentage, or the expression level between the two groups. Instead, a feature that stood out was the distance between the arms of an inverted Alu pair (i.e., the loop size). Immunogenic inverted Alus had significantly smaller loop sizes than their non-immunogenic counterparts.

To validate immunogenic dsRNAs as MDA5 substrates, we examined their ability to form MDA5 filaments in vitro using negative stain electron microscopy. We observed a correlation between dsRNA length and MDA5 filament length. We then chose to validate candidate dsRNAs, representing inverted Alu, endogenous retroviral (ERV) repeats as well as cis-NATs. In all cases, we observed filaments with varying lengths dependent on the length of the dsRNA.

Identification of immunogenic dsRNAs in human neural progenitor cells. Several lines of evidence suggest that there are different repertoires of immunogenic dsRNAs in different cell types, tissues, and organisms. Neural progenitor cells were used as a prototype. To identify immunogenic dsRNAs in NPCs, we performed a comparative editing analysis similar to what we described above. We stably integrated the ADAR1 p150 or p110 isoform into ADAR1−/− NPCs immediately after differentiation. As expected, p150, but not p110, suppressed ISG induction. We identified 950 (2.4%) immunogenic dsRNA candidates that were significantly more highly edited by p150 than p110.

Only a subset (165) of immunogenic dsRNAs were found in both HEK293T and NPCs.

When we confined our analysis to genes that are expressed in both cell types, the immunogenic dsRNA sets almost entirely overlapped. This suggests that the immunogenicity of a dsRNA is intrinsically determined by its sequence and structure features, and its effect is dependent on the host gene expression that may vary across cell and tissue types. This implies that the immunogenic dsRNAs identified in any cell types would be immunogenic in other cell types as long as they are expressed.

Immunogenic dsRNAs are enriched at the GWAS signals of common inflammatory diseases. We asked if immunogenic dsRNA candidates we identified would be enriched at the GWAS signals of common autoimmune and immune-related diseases. Indeed, we found that the immunogenic, but not the non-immunogenic dsRNA candidates, showed strong enrichment at the GWAS signals of inflammatory diseases, as exemplified in autoimmune thyroid disease, rheumatoid arthritis, type 1 diabetes, inflammatory bowel disease and others.

Example 1, above, discloses hundreds of immunogenic dsRNA candidates that are colocalized at the GWAS loci for autoimmune and immune-related diseases. While this computational approach is successful in identifying disease relevant dsRNAs, it may be complemented by an unbiased search of immunogenic dsRNAs in a given cell type.

### Methods

De novo identification of A-to-I RNA editing site. De novo identification of editing sites was performed on pooled mapping results of HEK293T and NPC samples. We used the UnifiedGenotyper tool from the Genome Analysis Toolkit (GATK)ₐₒ to call variants from mapped RNA-seq reads. We then applied parameters and filters as described previouslyzzzs. In brief, human variants at non-repetitive and repetitive non-Alu sites were required to be supported by at least three mismatch reads. A support of one mismatch read was required for variants in human Alu regions. This set of variant candidates was subject to several filtering steps that increased the accuracy of editing site discovery. We also removed all known SNPs present in dbSNP (except SNPs of molecular type “cDNA”; database version 135) the 1000 Genomes Project, and the University of Washington Exome Sequencing Project). We also performed hyper-editing identification to recover the editing sites from unmapped reads. At last, we combined known editing sites from RADAR and the newly identified sites from each cell line, and obtained 3,949,224 and 2,982,889 editing sites in HEK293T and NPC, respectively.

Identification of A-to-I RNA editing clusters. To identify the long dsRNAs that are potential MDA5 ligands without relying on any prior annotation, we first reasoned that these long dsRNAs should all be hyper-edited in order to suppress their immunogenicity. We took the location information of editing sites in the genome as proxy for dsRNAs and applied a density-based clustering algorithm (DBSCAN) to identify genomic regions with many editing sites close together, which we called editing clusters. DBSCAN requires two parameters to define a cluster: the minimum number of points (editing sites) required to form a cluster (minPts) and the farthest distance between two adjacent sites within the same cluster (eps). For the first parameter (minPts), as expected, we observed that the total number of clusters decreases as the minimum number of editing sites per cluster increases. We set minPts=5 as the minimum number of mismatches (edits). For the second parameter (eps), by plotting the distance between adjacent sites sorted from the nearest to the farthest, we observed a “knee” point on the curve, where eps =120. Since this “knee” point allows the inclusion of >90% editing sites in editing clusters while excluding the far away outliers, we concluded that eps=120 is the optimal value. By applying DBSCAN clustering, we identified 205,652 and 175,655 editing clusters in HEK293T and NPC cell lines, respectively. Further after applying the coverage cutoff (>20 reads), we obtained 39,079 and 40,182 editing clusters in HEK293T and NPC cell lines for downstream analyses, respectively.

Quantification and comparison of editing index. We adopted the concept of an “editing index” as the measurement of the editing level of an editing cluster. For each cluster containing n editing sites, its editing index is calculated as:

$$\frac{{\sum }_{1}^{n} ( G reads )}{{\sum }_{1}^{n} ( A + G reads )} .$$

To ensure the measurement of editing index is reliable, we required that:

$${\sum }_{1}^{n} ( A + G reads ) \geq 20 n .$$

For each editing cluster meeting the above threshold in both conditions in comparison, Fisher's exact test was applied to the counts of G and A reads to calculate the p-value for the significance of the editing indices not being equal. Multi-test p-values were adjusted using Benjamini-Hochberg procedure.

TABLE 2
Interferon-stimulated Genes

ABLIM3
ABTB2
ACSL1
ADAMDEC1
ADAR
ADM
AHNAK2
AIM2
AKT3
ALDH1A1
ALYREF
AMPH
ANGPTL1
ANKFY1
ANKRD22
ANXA2R
APOBEC3A
APOL1
APOL2
AQP9
ARG2
ARHGEF3
ARNTL
ATF3
ATP10D
B2M
B4GALT5
BAG1
BATF2
BCL2L14
BCL3
BLVRA
BST2
BTN3A3
BUB1
C10orf10
C15orf48
C1S
C22orf28
C4orf32
C4orf33
C5orf27
C9orf91
CASP7
CCDC109B
CCDC92
CCL19
CCL2
CCL4
CCL5
CCL8
CCNA1
CCND3
CCR1
CD163
CD274
CD38
CD69
CD74
CD80
CD9
CDK17
CDKN1A
CEBPD
CES1
CFB
CHMP5
CLEC2B
CLEC4D
CLEC4E
CMAHP
COMMD3
CPT1A
CREB3L3
CRP
CRY1
CSRNP1
CTCFL
CX3CL1
CXCL10
CXCL11
CXCL9
CYP1B1
CYTH1
DCP1A
DDIT4
DDX3X
DDX58
DDX60
DTX3L
DUSP5
EHD4
EIF3L
ELF1
ENPP1
EPSTI1
ERLIN1
ETV6
ETV7
EXT1
FAM134B
FAM46A
FCGR1A
FFAR2
FKBP5
FNDC3B
FNDC4
FTSJD2
FUT4
FZD5
G6PC
GAK
GALNT2
GBA
GBP1
GBP2
GBP3
GBP4
GBP5
GCA
GCH1
GEM
GJA4
GK
GLRX
GPX2
GZMB
HEG1
HELZ2
HERC6
HES4
HESX1
HLA-A
HLA-B
HLA-C
HLA-E
HLA-F
HLA-G
HSH2D
IDO1
IFI16
IFI27
IFI30
IFI35
IFI44
IFI44L
IFI6
IFIT1
IFIT2
IFIT3
IFIT5
IFITM2
IFITM3
IFNGR1
IFNLR1
IGFBP2
IL15
IL15RA
IL17RB
IL1R1
IL1RN
IL6ST
IMPA2
IRF2
IRF7
IRF9
ISG15
ISG20
JAK2
JUNB
KIAA0040
LAMP3
LAP3
LEPR
LGALS3
LGMN
LIPA
LMO2
LPCAT1
LRG1
LY6E
MAB21L2
MAFB
MAFF
MAP3K14
MAP3K5
MARCKS
MASTL
MAX
MB21D1
MCL1
MCOLN2
MICB
MKX
MSR1
MT1F
MT1G
MT1H
MT1M
MT1X
MTHFD2L
MVB12B
MX1
MYD88
MYOF
NAMPT
NAPA
NCF1
NCOA3
NDC80
NEURL3
NFIL3
NMI
NOD2
NPAS2
NRN1
NT5C3
OAS2
OAS3
OASL
ODC1
OGFR
OPTN
P2RY6
PADI2
PARP12
PARP9
PDGFRL
PDK1
PFKFB3
PHF11
PHF15
PI4K2B
PIM3
PLIN2
PLSCR1
PMAIP1
PML
PMM2
PNPT1
PNRC1
PPM1K
PRKD2
PSMB8
PSMB9
PTMA
PUS1
PXK
RARRES3
RASSF4
RBCK1
RBM25
RGS1
RNASE4
RNF114
RNF19B
RNF213
RPL22
RTP4
S100A8
SAA1
SAMD4A
SAMHD1
SCARB2
SCO2
SECTM1
SERPINB9
SERPINE1
SERPING1
SIRPA
SLC15A3
SLC16A1
SLC1A1
SLC25A28
SLC25A30
SLFN5
SMAD3
SNN
SOCS1
SOCS2
SP110
SPATS2L
SPSB1
SPTLC2
ST3GAL4
STAP1
STARD5
STAT1
STEAP4
SUN2
TAGAP
TAP1
TAP2
TBX3
TCF7L2
TDRD7
TFEC
THBD
TIMP1
TLK2
TLR3
TMEM140
TMEM255A
TMEM51
TNFAIP3
TNFAIP6
TNFRSF10A
TNFSF10
TNFSF13B
TRAFD1
TREX1
TRIM14
TRIM21
TRIM25
TRIM34
TRIM38
TRIM5
TXNIP
TYMP
UBE2L6
ULK4
UNC93B1
UPP2
USP18
VAMP5
VEGFC
VMP1
WARS
WHAMM
XAF1
ZBP1
ZNF385B

### REFERENCES

Albert, F. W. & Kruglyak, L. The role of regulatory variation in complex traits and disease. Nat Rev Genet 16, 197-212, doi: 10.1038/nrg3891 (2015).
Consortium, G. T. The GTEx Consortium atlas of genetic regulatory effects across human tissues. Science 369, 1318-1330, doi: 10.1126/science.aaz1776 (2020).
Chun, S. et al. Limited statistical evidence for shared genetic effects of eQTLs and autoimmune-disease-associated loci in three major immune-cell types. Nat Genet 49, 600-605, doi: 10.1038/ng.3795 (2017).
Umans, B. D., Battle, A. & Gilad, Y. Where Are the Disease-Associated eQTLs? Trends Genet 37, 109-124, doi: 10.1016/j.tig.2020.08.009 (2021).
Nishikura, K. Functions and regulation of RNA editing by ADAR deaminases. Annual review of biochemistry 79, 321-349, doi: 10.1146/annurev-biochem-060208-105251 (2010).
Rice, G. I. et al. Mutations in ADAR1 cause Aicardi-Goutieres syndrome associated with a type I interferon signature. Nat Genet 44, 1243-1248, doi: 10.1038/ng.2414 (2012).
Mannion, N. M. et al. The RNA-editing enzyme ADAR1 controls innate immune responses to RNA. Cell reports 9, 1482-1494, doi: 10.1016/j.celrep.2014.10.041 (2014).
Liddicoat, B. J. et al. RNA editing by ADAR1 prevents MDA5 sensing of endogenous dsRNA as nonself. Science 349, 1115-1120, doi: 10.1126/science.aac7049 (2015).
Pestal, K. et al. Isoforms of RNA-Editing Enzyme ADAR1 Independently Control Nucleic Acid Sensor MDA5-Driven Autoimmunity and Multi-organ Development. Immunity 43, 933-944, doi: 10.1016/j.immuni.2015.11.001 (2015).
Eisenberg, E. & Levanon, E. Y. A-to-I RNA editing-immune protector and transcriptome diversifier. Nat Rev Genet 19, 473-490, doi: 10.1038/s41576-018-0006-1 (2018).
Samuel, C. E. Adenosine deaminase acting on RNA (ADAR1), a suppressor of double-stranded RNA-triggered innate immune responses. The Journal of biological chemistry 294, 1710-1720, doi: 10.1074/jbc.TM118.004166 (2019).
Li, Y. I. et al. RNA splicing is a primary link between genetic variation and disease. Science 352, 600-604, doi: 10.1126/science.aad9417 (2016).
Jonkhout, N. et al. The RNA modification landscape in human disease. RNA 23, 1754-1769, doi: 10.1261/rna.063503.117 (2017).
Walkley, C. R. & Li, J. B. Rewriting the transcriptome: adenosine-to-inosine RNA editing by ADARs. Genome Biol 18, 205, doi: 10.1186/s13059-017-1347-3 (2017).
Ramaswami, G. & Li, J. B. Identification of human RNA editing sites: A historical perspective. Methods 107, 42-47, doi: 10.1016/j.ymeth.2016.05.011 (2016).
Ramaswami, G. et al. Accurate identification of human Alu and non-Alu RNA editing sites. Nat Methods 9, 579-581, doi: 10.1038/nmeth. 1982 (2012).
Tan, M. H. et al. Dynamic landscape and regulation of RNA editing in mammals. Nature 550, 249-254, doi: 10.1038/nature24041 (2017).
Porath, H. T., Knisbacher, B. A., Eisenberg, E. & Levanon, E. Y. Massive A-to-I RNA editing is common across the Metazoa and correlates with dsRNA abundance. Genome Biol 18, 185, doi: 10.1186/s13059-017-1315-y (2017).
Chalk, A. M., Taylor, S., Heraud-Farlow, J. E. & Walkley, C. R. The majority of A-to-I RNA editing is not required for mammalian homeostasis. Genome Biol 20, 268, doi: 10.1186/s 13059-019-1873-2 (2019).
Rice, G. I. et al. Gain-of-function mutations in IFIH1 cause a spectrum of human disease phenotypes associated with upregulated type I interferon signaling. Nat Genet 46, 503-509, doi: 10.1038/ng.2933 (2014).
Nejentsev, S., Walker, N., Riches, D., Egholm, M. & Todd, J. A. Rare variants of IFIH1, a gene implicated in antiviral responses, protect against type 1 diabetes. Science 324, 387-389, doi: 10.1126/science. 1167728 (2009).
Emdin, C. A. et al. Analysis of predicted loss-of-function variants in UK Biobank identifies variants protective for disease. Nature communications 9, 1613, doi: 10.1038/s41467-018-03911-8 (2018). Li, Y. et al. Carriers of rare missense variants in IFIH1 are protected from psoriasis. J Invest Dermatol 130, 2768-2772, doi: 10.1038/jid.2010.214 (2010).
Huang, H. et al. Fine-mapping inflammatory bowel disease loci to single-variant resolution. Nature 547, 173-178, doi: 10.1038/nature22969 (2017).
Jin, Y., Andersen, G. H. L., Santorico, S. A. & Spritz, R. A. Multiple Functional Variants of IFIH1, a Gene Involved in Triggering Innate Immune Responses, Protect against Vitiligo. J Invest Dermatol 137, 522-524, doi: 10.1016/j.jid.2016.09.021 (2017).
Sun, B. B. et al. Genetic associations of protein-coding variants in human disease. Nature, doi: 10.1038/s41586-022-04394-w (2022).
Shallev, L. et al. Decreased A-to-I RNA editing as a source of keratinocytes' dsRNA in psoriasis. RNA 24, 828-840, doi: 10.1261/rna.064659.117 (2018).
Vlachogiannis, N. I. et al. Increased adenosine-to-inosine RNA editing in rheumatoid arthritis. J Autoimmun 106, 102329, doi: 10.1016/j.jaut.2019.102329 (2020).
Roth, S. H. et al. Increased RNA Editing May Provide a Source for Autoantigens in Systemic Lupus Erythematosus. Cell reports 23, 50-57, doi: 10.1016/j.celrep.2018.03.036 (2018).
Tossberg, J. T., Heinrich, R. M., Farley, V. M., Crooke, P. S., 3rd & Aune, T. M. Adenosine-to-Inosine RNA Editing of Alu Double-Stranded (ds) RNAs Is Markedly Decreased in Multiple Sclerosis and Unedited Alu dsRNAs Are Potent Activators of Proinflammatory Transcriptional Responses. J Immunol 205, 2606-2617, doi: 10.4049/jimmunol.2000384 (2020).
Belkadi, A. et al. Identification of genetic variants controlling RNA editing and their effect on RNA structure stabilization. Eur J Hum Genet 28, 1753-1762, doi: 10.1038/s41431-020-0688-7 (2020). Ruan, H. et al. GPEdit: the genetic and pharmacogenomic landscape of A-to-I RNA editing in cancers. Nucleic Acids Res 50, D1231-D1237, doi: 10.1093/nar/gkab810 (2022).
Breen, M. S. et al. Global landscape and genetic regulation of RNA editing in cortical samples from individuals with schizophrenia. Nat Neurosci 22, 1402-1412, doi: 10.1038/s41593-019-0463-7 (2019).
Park, E., Jiang, Y., Hao, L., Hui, J. & Xing, Y. Genetic variation and microRNA targeting of A-to-I RNA editing fine tune human tissue transcriptomes. Genome Biol 22, 77, doi: 10.1186/s13059-021-02287-1 (2021).
Franzen, O. et al. Global analysis of A-to-I RNA editing reveals association with common disease variants. PeerJ 6, e4466, doi: 10.7717/peerj.4466 (2018).
Park, E. et al. Population and allelic variation of A-to-I RNA editing in human transcriptomes. Genome Biol 18, 143, doi: 10.1186/s13059-017-1270-7 (2017).
Wijetunga, N. A. et al. The meta-epigenomic structure of purified human stem cell populations is defined at cis-regulatory sequences. Nature communications 5, 5195, doi: 10.1038/ncomms6195 (2014).
Baeza-Centurion, P., Minana, B., Schmiedel, J. M., Valcarcel, J. & Lehner, B. Combinatorial Genetics Reveals a Scaling Law for the Effects of Mutations on Splicing. Cell 176, 549-563 e523, doi: 10.1016/j.cell.2018.12.010 (2019).
Ongen, H., Buil, A., Brown, A. A., Dermitzakis, E. T. & Delaneau, O. Fast and efficient QTL mapper for thousands of molecular phenotypes. Bioinformatics 32, 1479-1485, doi: 10.1093/bioinformatics/btv722 (2016).
Urbut, S. M., Wang, G., Carbonetto, P. & Stephens, M. Flexible statistical methods for estimating and testing effects in genomic studies with multiple conditions. Nat Genet 51, 187-195, doi: 10.1038/s41588-018-0268-8 (2019).
Matthews, M. M. et al. Structures of human ADAR2 bound to dsRNA reveal base-flipping mechanism and basis for site selectivity. Nat Struct Mol Biol 23, 426-433, doi: 10.1038/nsmb.3203 (2016).
Hormozdiari, F. et al. Leveraging molecular quantitative trait loci to understand the genetic architecture of diseases and complex traits. Nat Genet 50, 1041-1047, doi: 10.1038/s41588-018-0148-2 (2018).
Finucane, H. K. et al. Partitioning heritability by functional annotation using genome-wide association summary statistics. Nat Genet 47, 1228-1235, doi: 10.1038/ng.3404 (2015).
Navab, M., Anantharamaiah, G. M., Reddy, S. T., Van Lenten, B. J. & Fogelman, A. M. HDL as a biomarker, potential therapeutic target, and therapy. Diabetes 58, 2711-2717, doi: 10.2337/db09-0538 (2009).
Zeisbrich, M. et al. Hypermetabolic macrophages in rheumatoid arthritis and coronary artery disease due to glycogen synthase kinase 3b inactivation. Ann Rheum Dis 77, 1053-1062, doi: 10.1136/annrheumdis-2017-212647 (2018).
Misra, M. K., Damotte, V. & Hollenbach, J. A. The immunogenetics of neurological disease. Immunology 153, 399-414, doi: 10.1111/imm. 12869 (2018).
Suciu, C. F. et al. Oxidized low density lipoproteins: The bridge between atherosclerosis and autoimmunity. Possible implications in accelerated atherosclerosis and for immune intervention in autoimmune rheumatic disorders. Autoimmun Rev 17, 366-375, doi: 10.1016/j.autrev.2017.11.028 (2018).
Yao, D. W., O'Connor, L. J., Price, A. L. & Gusev, A. Quantifying genetic effects on disease mediated by assayed gene expression levels. Nat Genet 52, 626-633, doi: 10.1038/s41588-020-0625-2 (2020).
Sayaman, R. W. et al. Germline genetic contribution to the immune landscape of cancer. Immunity 54, 367-386 e368, doi: 10.1016/j.immuni.2021.01.011 (2021).
Bazak, L. et al. A-to-I RNA editing occurs at over a hundred million genomic sites, located in a majority of human genes. Genome Res 24, 365-376, doi: 10.1101/gr. 164749.113 (2014).
Faghihi, M. A. & Wahlestedt, C. Regulatory roles of natural antisense transcripts. Nat Rev Mol Cell Biol 10, 637-643, doi: 10.1038/nrm2738 (2009).
Dias Junior, A. G., Sampaio, N. G. & Rehwinkel, J. A Balancing Act: MDA5 in Antiviral Immunity and Autoinflammation. Trends Microbiol 27, 75-85, doi: 10.1016/j.tim.2018.08.007 (2019).
Wu, B. et al. Structural basis for dsRNA recognition, filament formation, and antiviral signal activation by MDA5. Cell 152, 276-289, doi: 10.1016/j.cell.2012.11.048 (2013).
Li, P., Zhou, X., Xu, K. & Zhang, Q. C. RASP: an atlas of transcriptome-wide RNA secondary structure probing data. Nucleic Acids Res 49, D183-D191, doi: 10.1093/nar/gkaa880 (2021).
Zhao, W. et al. POSTAR3: an updated platform for exploring post-transcriptional regulation coordinated by RNA-binding proteins. Nucleic Acids Res 50, D287-D294, doi: 10.1093/nar/gkab702 (2022).
Reshef, Y. A. et al. Detecting genome-wide directional effects of transcription factor binding on polygenic disease risk. Nat Genet 50, 1483-1493, doi: 10.1038/s41588-018-0196-7 (2018).
Patterson, J. B. & Samuel, C. E. Expression and regulation by interferon of a double-stranded-RNA-specific adenosine deaminase from human cells: evidence for two forms of the deaminase. Mol Cell Biol 15, 5376-5388, doi: 10.1128/mcb. 15.10.5376 (1995).
Heraud-Farlow, J. E. & Walkley, C. R. The role of RNA editing by ADAR1 in prevention of innate immune sensing of self-RNA. J Mol Med (Berl) 94, 1095-1102, doi: 10.1007/s00109-016-1416-1 (2016).
Guo, Y. et al. CD40L-Dependent Pathway Is Active at Various Stages of Rheumatoid Arthritis Disease Progression. J Immunol 198, 4490-4501, doi: 10.4049/jimmunol. 1601988 (2017).
Elkjaer, M. L. et al. Molecular signature of different lesion types in the brain white matter of patients with progressive multiple sclerosis. Acta Neuropathol Commun 7, 205, doi: 10.1186/s40478-019-0855-7 (2019).
Tokuyama, M. et al. ERVmap analysis reveals genome-wide transcription of human endogenous retroviruses. Proc Natl Acad Sci USA 115, 12565-12572, doi: 10.1073/pnas. 1814589115 (2018). Numata, K. et al. Comparative analysis of cis-encoded antisense RNAs in eukaryotes. Gene 392, 134-141, doi: 10.1016/j.gene.2006.12.005 (2007).
Ramaswami, G. & Li, J. B. RADAR: a rigorously annotated database of A-to-I RNA editing. Nucleic Acids Res 42, D109-113, doi: 10. 1093/nar/gkt996 (2014).
Zhang, R. et al. Quantifying RNA allelic ratios by microfluidic multiplex PCR and sequencing. Nat Methods 11, 51-54, doi: 10.1038/nmeth.2736 (2014).
Ramaswami, G. et al. Genetic mapping uncovers cis-regulatory landscape of RNA editing. Nature communications 6, 8194, doi: 10.1038/ncomms9194 (2015).
Tran, S. S. et al. Widespread RNA editing dysregulation in brains from autistic individuals. Nat Neurosci 22, 25-36, doi: 10.1038/s41593-018-0287-x (2019).
Storey, J. D. & Tibshirani, R. Statistical significance for genomewide studies. Proc Natl Acad Sci USA 100, 9440-9445, doi: 10.1073/pnas. 1530509100 (2003).
Frankish, A. et al. GENCODE reference annotation for the human and mouse genomes. Nucleic Acids Res 47, D766-D773, doi: 10.1093/nar/gky955 (2019).
Bahn, J. H. et al. Genomic analysis of ADAR 1 binding and its involvement in multiple RNA processing pathways. Nature communications 6, 6355, doi: 10.1038/ncomms7355 (2015).
Dobin, A. et al. STAR: ultrafast universal RNA-seq aligner. Bioinformatics 29, 15-21, doi: 10.1093/bioinformatics/bts635 (2013).
Altschul, S. F., Gish, W., Miller, W., Myers, E. W. & Lipman, D. J. Basic local alignment search tool. J Mol Biol 215, 403-410, doi: 10.1016/S0022-2836 (05) 80360-2 (1990).
Konkel, M. K. et al. Sequence Analysis and Characterization of Active Human Alu Subfamilies Based on the 1000 Genomes Pilot Project. Genome biology and evolution 7, 2608-2622, doi: 10.1093/gbe/evv167 (2015).
Eggington, J. M., Greene, T. & Bass, B. L. Predicting sites of ADAR editing in double-stranded RNA. Nature communications 2, 319, doi: 10.1038/ncomms1324 (2011).
Wagih, O. ggseqlogo: a versatile R package for drawing sequence logos. Bioinformatics 33, 3645-3647, doi: 10.1093/bioinformatics/btx469 (2017).
Tian, S. & Das, R. RNA structure through multidimensional chemical mapping. Q Rev Biophys 49, e7, doi: 10.1017/S0033583516000020 (2016).
Danaee, P. et al. bpRNA: large-scale automated annotation and analysis of RNA secondary structure. Nucleic Acids Res 46, 5381-5394, doi: 10. 1093/nar/gky285 (2018).
Purcell, S. et al. PLINK: a tool set for whole-genome association and population-based linkage analyses. Am J Hum Genet 81, 559-575, doi: 10.1086/519795 (2007).
Pers, T. H., Timshel, P. & Hirschhorn, J. N. SNPsnap: a Web-based tool for identification and annotation of matched SNPs. Bioinformatics 31, 418-420, doi: 10. 1093/bioinformatics/btu655 (2015).
Gazal, S. et al. Linkage disequilibrium-dependent architecture of human complex traits shows action of negative selection. Nat Genet 49, 1421-1427, doi: 10.1038/ng.3954 (2017).
Rice, G. I. et al. Assessment of Type I Interferon Signaling in Pediatric Inflammatory Disease. J Clin Immunol 37, 123-132, doi: 10.1007/s10875-016-0359-1 (2017).
Giambartolomei, C. et al. Bayesian test for colocalisation between pairs of genetic association studies using summary statistics. PLOS Genet 10, e1004383, doi: 10.1371/journal.pgen. 1004383 (2014).
Balbin, O. A. et al. The landscape of antisense gene expression in human cancers. Genome Res 25, 1068-1079, doi: 10.1101/gr. 180596.114 (2015).
Lorenz, R. et al. ViennaRNA Package 2.0. Algorithms Mol Biol 6, 26, doi: 10.1186/1748-7188 Jun. 26 (2011).
Zhang, F., Lu, Y., Yan, S., Xing, Q. & Tian, W. SPRINT: an SNP-free toolkit for identifying RNA editing sites. Bioinformatics 33, 3538-3548, doi: 10.1093/bioinformatics/btx473 (2017).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for determining an immunogenic double-stranded RNA (dsRNA) burden score of an individual, the method comprising:
genotyping said subject at a plurality of risk alleles associated with RNA editing level changes for an immune-related disease of interest;
generating an immunogenic dsRNA burden score by weighting the number of risk alleles that decrease RNA editing levels by combining 1) the effect size of a risk allele association with the disease of interest, 2) the effect sizes of a risk allele association with RNA editing level, 3) the expression levels of immunogenic dsRNAs in disease relevant tissues or cells to generate a dsRNA burden score;
wherein the dsRNA burden score is associated with an individual's predisposition to the disease of interest due to unwanted activation of IFN immune response and responsiveness to therapy that reduces the clinical sequelae of undesirable activity in dsRNA sensing pathways.

2. The method of claim 1 wherein at least 100, at least 200, at least 500, at least 1000, at least 5000, at least 10,000, at least 15,000, at least 20,000, at least 25,000, at least 30,000 SNPs are genotyped.

3. The method of claim 1, further comprising genotyping the individual at one or more alleles associated with MDA5 or ADAR1.

4. The method of claim 1, wherein the disease of interest affects a specific tissue or cell type of interest.

5. The method of claim 1, further comprising determining the IFN response level and expression level of dsRNAs in the tissue of interest or cells derived therefrom.

6. The method of claim 1, wherein the dsRNAs are selected by computational colocalization at GWAS loci of diseases of interest.

7. The method of claim 1, wherein dsRNAs are determined by identifying editing clusters that have least five editing sites and any two adjacent sites are less than 120 nt apart;
determining editing clusters whose editing index is significantly higher in ADAR1 p150-than ADAR1 p110-complemented cells; where editing clusters preferably edited by p150 are selected as immunogenic dsRNA candidates.

8. The method of claim 1, wherein the immunogenic dsRNA score further comprises determining tissues or cells of disease relevance and expression level of immunogenic dsRNAs derived therefrom.

9. The method of claim 8, wherein tissues or cells of disease relevance are selected by (a) measuring interferon (IFN) score by aggregated expression levels of interferon-stimulated genes (ISGs) to determine the highest tissue or cell, and (b) measuring the aggregated expression level of immunogenic dsRNAs in disease tissue scRNA-seq.

10. The method of claim 1 wherein the expression level of at least 100, at least 200, at least 500, at least 1000, at least 5000, at least 10,000 dsRNA loci is determined.

11. The method of claim 1, further comprising inputting additional clinical indicia of age, gender, family history, age of onset of said disease, duration of said disease.

12. The method of claim 1, wherein a pre-determined threshold for the score is set by training on genome-wide association study to assess distribution of IDS between cases and controls for the disease of interest to determine the IDS threshold for a prediction of high risk for the disease.

13. The method claim 1, wherein the individual is previously diagnosed with the disease of interest.

14. The method claim 1, wherein the disease of interest is an autoimmune disease.

15. The method claim 1, wherein the disease of interest is an inflammatory disease.

16. The method of claim 1, wherein the disease of interest is selected from autoimmune thyroid disease, celiac disease, inflammatory bowel disease, systemic lupus erythematosus, multiple sclerosis, primary biliary cirrhosis, psoriasis, type 1 diabetes (T1D), rheumatoid arthritis, vitiligo; for atopic conditions such as asthma and atopic dermatitis; and for other conditions with a significant inflammatory component, e.g. amyotrophic lateral sclerosis (ALS), coronary artery disease, type 2 diabetes, Parkinsons disease, systemic sclerosis, schizophrenia, Alzheimers disease, and levels of high and low density lipoproteins and triglycerides.

17. The method of claim 1, wherein the determination of a dsRNA burden score is performed as a program of instructions executable by computer and performed by means of software components loaded into the computer.

18. The method of claim 1, further comprising treating the individual is accordance with the determination of dsRNA burden score.

19. The method of claim 1, wherein the immunogenic dsRNA burden score (Y) is determined by the equation
$${Ψ}^{k} = {{{\sum }^{n}}_{i = 1 , j = 1}}_{} {β}_{j}^{k} \times {ϕ}_{ij}^{k} \times {ε}_{i}^{k}$$
where
$${β}_{j}^{k}$$
is the effect size of j^{th} SNP for the disease in k^{th} tissue/cell type,
$${ϕ}_{ij}^{k}$$
is the effect size of i^{th} dsRNA with j^{th} SNP for RNA editing level in k^{th} tissue/cell type and
$${ε}_{ij}^{k}$$
is the expression level of i^{th} dsRNA in k^{th} tissue/cell type.

20. The method of claim 19, wherein the dsRNA burden score further comprises one or more clinical components, optionally wherein said dsRNA burden score identifies an individual as responsive to MDA5-inhibition therapy, optionally wherein the dsRNA burden score identifies an individual with increased MDA5-mediated immune response due to lack of dsRNA editing in a tissue of interest

21-22. (canceled)
